(19) United States
(12) Patent Application Publication
(10) Pub. No.: US 2003/0235556 A1
(43) Pub. Date: Dec. 25, 2003

(54) Combination IL-2/anti-HER2 antibody therapy for cancers characterized by overexpression of the HER2 receptor protein

(75) Inventors: Maurice J. Wolin, Piedmont, CA (US); Sandra Milan, Orinda, CA (US)

Correspondence Address: Chiron Corporation, 4560 Horton Street, Emeryville, CA, 94608, US

(73) Assignee: Chiron Corporation, Emeryville, CA

(21) Appl. No.: 10/346,468

(22) Filed: Jan. 18, 2003

Related U.S. Application Data
(60) Provisional application No. 60/349,158, filed on Jan. 18, 2002.

(51) Int. Cl. A61K 39/395; A61K 38/20

(52) U.S. Cl. 424/85.2; 424/143.1

(57) ABSTRACT

Methods for treating a subject with a cancer that is characterized by overexpression of HER2 receptor protein using a combination of interleukin-2 (IL-2) or biologically active variant thereof and at least one anti-HER2 antibody or antigen-binding fragment thereof are provided. These therapeutic agents are administered as two separate pharmaceutical compositions, one containing IL-2 (or variant thereof), which is administered according to a constant IL-2 dosing regimen or a two-level IL-2 dosing regimen, the other containing at least one anti-HER2 antibody (or fragment thereof), which is administered according to a weekly dosing regimen, or is administered once every two, three, or four weeks. Administering of these two agents together potentiates the effectiveness of the anti-HER2 antibody alone, resulting in a positive therapeutic response that is improved with respect to that observed with this therapeutic agent alone.

### CROSS REFERENCE TO RELATED APPLICATION

This application claims the benefit of U.S. Provisional Application No. 60,349,158, filed Jan. 18, 2002, the content of which is herein incorporated by reference in its entirety.

### FIELD OF THE INVENTION

The present invention is directed to methods of therapy for cell proliferative disorders, more particularly to concurrent therapy with interleukin-2 and monoclonal antibodies targeting the HER2 receptor protein to treat cancers characterized by overexpression of the HER2 receptor protein.

### BACKGROUND OF THE INVENTION

Cancer research has turned to the use of monoclonal antibodies as therapeutic agents. Produced in a similar fashion to diagnostic antibodies, therapeutic antibodies are designed to target tumor cells in order to facilitate their destruction. The use of therapeutic monoclonal antibodies has been hampered in the past primarily because of issues related to the antigenicity of the protein. Monoclonal antibodies have traditionally been a mouse product, and therefore generate an anti-murine response when injected into humans. This so-called HAMA (human anti-mouse antibody) response has imposed a great limitation on the use of monoclonal antibodies, as repeated dosing is nearly always precluded. In addition, serious complications, such as serum sickness, have been reported with the use of these agents. With the advent of chimeric and humanized antibodies, the therapeutic benefit of monoclonal antibodies is being realized. Using recombinant DNA technology, it is possible for a monoclonal antibody to be constructed by joining the variable or antigen recognition site of the antibody to a human backbone. This construction greatly decreases the incidence of blocking or clearing of the foreign antibodies from the host. This development allows for multiple doses of antibody to be given, providing the opportunity for reproducible and sustained responses with this therapy.

Recent cancer research has focused on the use of recombinant humanized monoclonal antibodies for the treatment of cancers whose cells overexpress the protein p185HER2. This 185-kDa growth factor receptor is encoded by the her-2 protooncogene, also referred to as neu and c-erbB-2 (Slamon et al. (1987) Science 235:177-182). The her-2 gene is closely related to, but distinct from, the gene encoding epidermal growth factor receptor (EGFR). Amplification of this gene has been linked to neoplastic transformation in human breast cancer cells (Slamon et al. (1987) supra). Overexpression of this protein has been identified within 20-30% of breast cancer patients, where it correlates with regionally advanced disease, increased probability of tumor recurrence, and reduced patient survival. As many as 30-40% of patients having gastric, endometrial, salivary gland, non-small cell lung, pancreatic, ovarian, peritoneal, prostate, or colorectal cancers may also exhibit overexpression of this protein.

The most widely recognized monoclonal antibody targeting HER2 receptor function is marketed under the tradename Herceptin® (commonly known as trastuzamab and available from Genentech, Inc., San Francisco, Calif.). This recombinant humanized monoclonal antibody has high affinity for p185HER2. Early clinical trials with patients having extensive metastatic breast carcinomas demonstrate the ability of this monoclonal antibody to inhibit growth of breast cancer cells that overexpress HER2 (Baselga et al. (1996) J. Clin. Oncol. 14(3):737-744). In one such trial, monotherapy with Herceptin® in metastatic breast cancer patients yielded an overall response rate of 14% (2% complete responders and 12% partial responders). The median duration of response was 9.1 months, median survival was 12.8 months (ranging from 0.5 to 24+ months). Twenty-four percent of the patients were progression free at 5.8 months (Genentech, Inc., data on file). Degree of overexpression of p185HER2 was predictive of treatment effect. In another clinical trial, monotherapy with Herceptin® yielded objective responses in 5 out of 43 assessable metastatic breast cancer patients (11.6%) (as cited in “Cancer and Leukemia Group B (CALGB) 9661, A Pilot Study of Low-dose Interleukin-2 plus Recombinant Human Anti-HER2 Monoclonal Antibody in Solid Tumors”; herein incorporated by reference).

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al. (1987) N. Engl. J. Med. 316:889-897; Rosenberg (1988) Ann. Surg. 208:121-135; Topalian et al. (1988) J. Clin. Oncol. 6:839-853; Rosenberg et al. (1988) N. Engl. J. Med. 319:1676-1680; and Weber et al. (1992) J. Clin. Oncol. 10:33-40). Although the anti-tumor activity of IL-2 has best been described in patients with metastatic melanoma and renal cell carcinoma, other diseases, notably lymphoma, also appear to respond to treatment with IL-2. However, high doses of IL-2 used to achieve positive therapeutic results with respect to tumor growth frequently cause severe side effects, including capillary leak, hypotension, and neurological changes (see, for example, Duggan et al. (1992) J. Immunotherapy 12:115-122; Gisselbrecht et al. (1994) Blood 83:2081-2085; and Sznol and Parkinson (1994) Blood 83:2020-2022). Studies have shown that IL-2 augments antibody-dependent cellular cytotoxicity in vitro, and potential natural killer cell effectors may be expanded and activated in vivo with low dose IL-2 (Cancer Immunol. Immunother. 46(1998):318).

Although both of these agents exhibit promising anti-tumor activity, their therapeutic potential for cancer patients needs further examination. Cancers whose cells overexpress the HER2 receptor can be particularly recalcitrant to treatment. New methods of therapy that provide a more aggressive approach are needed.

### SUMMARY OF THE INVENTION

Methods for providing treatment to a subject with a cancer characterized by overexpression of the pl 85HER2 growth factor receptor using a combination of interleukin-2 or biologically active variant thereof (hereinafter collectively “IL-2”) and at least one anti-HER2 antibody or antigen-binding fragment thereof (hereinafter collectively “anti-HER2 antibody”) are provided. These two therapeutic agents are concurrently administered as two separate pharmaceutical compositions, one containing IL-2, the other containing at least one anti-HER2 antibody, each according to a particular dosing regimen. The pharmaceutical composition comprising the anti-HER2 antibody is administered according to a weekly dosing schedule, or alternatively is dosed once every two, three, or four weeks. The pharmaceutical composition comprising IL-2 is administered according to a constant IL-2 dosing regimen, or is administered according to a two-level IL-2 dosing regimen.

The constant IL-2 dosing regimen comprises a time period during which a constant total weekly dose of IL-2 is administered to the subject followed by a time period off of IL-2 dosing. One or more cycles of a constant IL-2 dosing regimen are administered to a subject in need thereof. The total weekly dose to be administered during each cycle of the constant IL-2 dosing regimen can be administered as a single dose. Alternatively, the total weekly dose administered during each cycle of the constant IL-2 dosing regimen can be partitioned into a series of equivalent doses that are administered according to a two-, three-, four-, five-, six- or seven-times-a-week dosing schedule.

The two-level IL-2 dosing regimen comprises a first time period of IL-2 dosing, wherein a higher total weekly dose of IL-2 is administered to the subject, followed by a second time period of IL-2 dosing, wherein a lower total weekly dose of IL-2 is administered to the subject. The total weekly dose of IL-2 during the second time period of IL-2 dosing is lower than the total weekly dose of IL-2 administered during the first time period of IL-2 dosing. The total weekly dose to be administered during the first time period and/or during the second time period of IL-2 dosing can be administered as a single dose. Alternatively, the total weekly dose administered during either or both of the first and second time periods of IL-2 dosing can be partitioned into a series of equivalent doses that are administered according to a two-, three-, four-, five-, six- or seven-times-a-week dosing schedule. The methods also provide for an interruption in the two-level dosing regimen of IL-2, where the subject is given a time period off of IL-2 administration between the first and second time periods of the two-level IL-2 dosing regimen. Concurrent therapy with these two therapeutic agents can comprise administering one or more cycles of a two-level IL-2 dosing regimen in combination with the recommended dosing regimen for the anti-HER2 antibody.

Administering of these two agents together in the manner set forth herein potentiates the effectiveness of the anti-HER2 antibody, resulting in a positive therapeutic response that is improved with respect to that observed with the antibody alone.

### DETAILED DESCRIPTION OF THE INVENTION

The present invention relates to methods of treating a human subject with a cancer characterized by overexpression of the pl 85HER2 growth factor receptor protein. The methods comprise combination therapy with interleukin-2 or biologically active variant thereof (hereinafter collectively “IL-2”) and at least one anti-HER2 antibody or antigen-binding fragment thereof (hereinafter collectively “anti-HER2 antibody”), each of which is administered according to a particular dosing regimen disclosed herein. These dosing regimens are followed until the subject is taken off anti-HER2 antibody therapy, for example, when the subject exhibits a complete response or exhibits one or more symptoms of anti-HER2 antibody toxicity as noted below, or is taken off IL-2 therapy due to development of IL-2 toxicity symptoms noted herein below.

Combination therapy with IL-2 and anti-HER2 antibody provides for anti-tumor activity. By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Therapy with a combination of IL-2 and at least one anti-HER2 antibody in the manner set forth herein causes a physiological response that is beneficial with respect to treatment of cancers whose unabated proliferating cells overexpress the HER2 receptor on their surface. Further, the particular IL-2 dosing regimens disclosed herein provide for intermittent stimulation of natural killer (NK) cell activity and decreased risk of IL-2-related side effects that can be associated with long-term exposure to IL-2 dosing.

p185HER2 is a 185 kDa cell-surface growth factor receptor protein that is a member of the tyrosine-specific protein kinase family to which many proto-oncogene products belong. The HER2 gene encoding this protein is also referred to in the art as the c-erB-2 gene. This human gene was reported by Semba et al. (1985) Proc. Natl. Acad. Sci. USA 82:6497-6501; Coussens et al. (1985) Science 230:1132-1139; and King et al (1985) Science 229:974-976. This proto-oncogene is a species variant of the rat neu gene, which was identified from chemically induced neuroblastomas (Schecter et al. (1985) Science 229:976-978). The HER2 protein encoded by HER2 has an extracellular domain, a transmembrane domain that includes two cysteine-rich repeat clusters, and an intracellular kinase domain, indicating that it is a cellular receptor for an as yet unidentified ligand. For purposes of the present invention, this growth factor receptor protein will hereinafter be referred to as HER2.

A small amount of HER2 protein is expressed on the plasma membrane of normal cells in a tissue-specific manner. This protein is present as part of a heterodimer receptor complex that binds a growth factor ligand. Binding of this ligand activates the HER2 receptor, resulting in the transmission of growth signals from the outside of the cell to the nucleus. These growth signals regulate aspects of normal cell growth and division. Alterations of the HER2 gene in normal cells leads to overexpression of the HER2 protein, resulting in increased cell division, increased rate of cell growth, and may be associated with transformation to a cancer cell phenotype. When such alterations in the HER2 gene occur in tumor cells, either the HER2 protein is directly overexpressed, or gene amplification results in multiple copies of the gene and subsequent overexpression of the HER2 protein. The factor(s) triggering these alterations are unknown at present.

By “overexpression” of the HER2 receptor protein is intended an abnormal level of expression of the HER2 receptor protein in a cell from a tumor within a specific tissue or organ of the patient relative to the level of expression in a normal cell from that tissue or organ. Patients having a cancer characterized by overexpression of the HER2 receptor can be determined by standard assays known in the art. Preferably overexpression is measured in fixed cells of frozen or paraffin-embedded tissue sections using immunohistochemical (IHC) detection. When coupled with histological staining, localization of the targeted protein can be determined and extent of its expression within a tumor can be measured both qualitatively and semi-quantitatively. Such IHC detection assays are known in the art and include the Clinical Trial Assay (CTA), the commercially available LabCorp 4D5 test, and the commercially available DAKO HercepTest™ (DAKO, Carpinteria, Calif.). The latter assay uses a specific range of 0 to 3+ cell staining (0 being normal expression, 3+indicating the strongest positive expression) to identify cancers having overexpression of the HER2 protein (see the Herceptin® (Trastuzumab) full prescribing information; September 1998; Genentech, Inc., San Francisco, Calif.). Thus, patients having a cancer characterized by overexpression by immunohistochemistry (IHC) or Fluorescent in-situ hybridization (FISH) of the HER2 protein in the range of 1+, 2+, or 3+, particularly 2+ or 3+, more particularly 3+, would benefit from the methods of therapy of the present invention.

Using standard detection assays, several types of cancers have been characterized as having cells that overexpress the HER2 receptor. Such cancers include, but are not limited to, breast, gastric, endometrial, salivary gland, non-small cell lung, pancreatic, renal, ovarian, peritoneal, prostate, bladder, colorectal cancers, and glioblastomas. Methods of the invention are useful in the treatment/management of any such cancer whose cells overexpress the HER2 receptor protein. Of particular interest is breast cancer. This is the most common malignancy among women in the United States, with 176,300 new cases projected for 1999 (Landis et al. (1999) CA Cancer J. Clin. 49:8-31). Overexpression of HER2 protein occurs in about 25-30% of all human breast cancers (Slamon et al. (1989) Science 244:707-712) and is associated with a poor clinical outcome (increased relapse and low survival rate), particularly in node-positive breast cancer patients.

While the methods of the invention are directed to treatment of an existing cancer, it is recognized that the methods may be useful in preventing further tumor outgrowths arising during therapy. The methods of the invention are particularly useful in the treatment of subjects having breast cancer, more particularly subjects having metastatic breast cancer and experiencing a relapse following one or more chemotherapy regimens for their metastatic disease, or whose prior treatment with anti-HER2 antibody failed. Thus, treatment of this type of cancer is improved using the methods of the invention, as the relative number of responders is increased.

In accordance with the methods of the present invention, IL-2 and at least one anti-HER2 antibody as defined elsewhere below are used in combination to promote a positive therapeutic response with respect to a cancer characterized by overexpression of the HER2 receptor protein. By “positive therapeutic response” is intended an improvement in the disease in association with the combined anti-tumor activity of these agents, and/or an improvement in the symptoms associated with the disease. Thus, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

Promotion of a positive therapeutic response in a subject with respect to a cancer characterized by overexpression of HER2 receptor is achieved via concurrent therapy with both IL-2 and at least one anti-HER2 antibody. By “concurrent therapy” is intended presentation of IL-2 and at least one anti-HER2 antibody to a subject in need thereof such that the therapeutic effect of the combination of both substances is caused in the subject undergoing therapy. Concurrent therapy may be achieved by administering at least one therapeutically effective dose of a pharmaceutical composition comprising IL-2 in accordance with a dosing schedule disclosed herein in combination with a pharmaceutical composition comprising at least one anti-HER2 antibody, where therapeutically effective amounts of the pharmaceutical composition comprising at least one anti-HER2 antibody are being administered in accordance with a recommended dosing regimen. For example, in accordance with the methods of the present invention, concurrent therapy is achieved by administering the recommended total weekly doses of a pharmaceutical composition comprising IL-2 in combination with the recommended therapeutically effective doses of a pharmaceutical composition comprising at least one anti-HER2 antibody, each being administered according to a particular dosing regimen. By “therapeutically effective dose or amount” is intended an amount of one of these two therapeutic agents that, when administered with a therapeutically effective dose or amount of the other of these two therapeutic agents, brings about a positive therapeutic response with respect to treatment of cancers characterized by overexpression of the HER2 receptor protein. Administration of the separate pharmaceutical compositions can be at the same time or at different times, so long as the therapeutic effect of the combination of both substances is caused in the subject undergoing therapy.

The separate pharmaceutical compositions comprising these therapeutic agents as therapeutically active components may be administered using any acceptable method known in the art. Thus, for example, the pharmaceutical composition comprising IL-2 can be administered by any form of injection, including intravenous (IV), intramuscular (IM), or subcutaneous (SC) injection. In some embodiments of the invention, the pharmaceutical composition comprising IL-2 is administered by SC injection. In other embodiments of the invention, the pharmaceutical composition comprising IL-2 is a sustained-release formulation, or a formulation that is administered using a sustained release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release IL-2 pharmaceutical composition. The pharmaceutical composition comprising the monoclonal antibody is administered, for example, intravenously. When administered intravenously, the pharmaceutical composition comprising the anti-HER2 antibody can be administered by infusion over a period of about 0.5 to about 5 hours. In some embodiments, infusion occurs over a period of about 0.5 to about 2.5 hours, over a period of about 0.5 to about 2.0 hours, over a period of about 0.5 to about 1.5 hours, or over a period of about 1.5 hours, depending upon the anti-HER2 antibody being administered and the amount of anti-HER2 antibody being administered.

Concurrent therapy with both of these therapeutic agents potentiates the anti-tumor activity of anti-HER2 antibody, thereby providing a positive therapeutic response that is improved with respect to that observed with therapy comprising administration of at least one anti-HER2 antibody alone. The amount of at least one anti-HER2 antibody to be administered in combination with an amount of IL-2, and the amount of either of these therapeutic agents needed to potentiate the effectiveness of the other therapeutic agent, are readily determined by one of ordinary skill in the art without undue experimentation given the disclosure set forth herein. Factors influencing the respective amount of IL-2 to be administered in combination with a given amount of at least one anti-HER2 antibody in accordance with the dosing regimens disclosed herein include, but are not limited to, the mode of administration, the particular cancer undergoing therapy, the severity of the disease, the history of the disease, and the age, height, weight, health, and physical condition of the individual undergoing therapy. Similarly, these factors will influence the necessity for repeated exposure to combination IL-2/anti-HER2 antibody therapy in the manner set forth herein. Generally, a higher dosage of the antibody agent is preferred with increasing weight of the subject undergoing therapy.

In accordance with the methods of the present invention, the human subject undergoing treatment with weekly doses of anti-HER2 antibody as defined herein below is also administered IL-2 as defined herein below according to a constant IL-2 dosing regimen or according to a two-level IL-2 dosing regimen. The first therapeutically effective dose administered to the subject can be the anti-HER2 antibody or can be the IL-2, depending upon which IL-2 dosing regimen is used.

Generally, where concurrent therapy with IL-2 and anti-HER2 antibody comprises one or more cycles of a constant IL-2 dosing regimen, the initial therapeutic agent to be administered to the subject at the start of a treatment period is the anti-HER2 antibody, while the first cycle of constant IL-2 dosing is initiated by administering a first dose of IL-2 subsequently, for example, within 10 days following administration of the first therapeutically effective dose of the anti-HER2 antibody, for example, within 1, 2, 3, 4, 5, 6, 7, 8, 9, or 10 days. In some embodiments, the first cycle of constant IL-2 dosing is initiated by administering a first dose of IL-2 within 7 days of administering the first therapeutically effective dose of anti-HER2 antibody, such as within 1, 2, 3, 4, 5, 6, or 7 days. Thus, for example, in one embodiment, a therapeutically effective dose of the anti-HER2 antibody is administered on day 1 of a treatment period, and the first cycle of constant IL-2 dosing is initiated 7 days later, i.e., by administering the initial dose of IL-2 on day 8 of the treatment period.

Following completion of the first cycle of constant IL-2 dosing, a human subject that is receiving weekly therapeutically effective doses of the anti-HER antibody can be administered one or more subsequent cycles of constant IL-2 dosing. During the second and all subsequent cycles of constant IL-2 dosing, generally the first therapeutic agent to be administered to the subject is the anti-HER2 antibody, with the second or subsequent cycle of constant IL-2 dosing being initiated by administering a first dose of IL-2 within 24 hours, such as within 0.5, 1, 2, 4, 8, 12, 16, 20, or 24 hours of administering the dose of anti-HER2 antibody. On those days where both anti-HER2 antibody and IL-2 are scheduled to be administered to the subject, these therapeutic agents can be administered either at the same time (i.e., simultaneous administration) or at different times (i.e., sequential administration, in either order). In one such embodiment, the dose of anti-HER2 antibody is administered first, followed by administration of the dose of IL-2 within about 10 minutes to about 4 hours of the completion of administering the dose of anti-HER2 antibody, such as within about 10, 15, 20, 25, 30, 45, 60, 90, 120, 150, 180, 210, or 240 minutes.

Where the subject in need of treatment is to receive a two-level IL-2 dosing regimen, either therapeutic agent can be administered first, so long as the subject has an overlapping period of time during which both therapeutic agents are being administered to the subject, each according to the particular dosing regimen disclosed herein.

Thus, in one embodiment, the two-level IL-2 dosing regimen is initiated prior to initiating weekly administration of therapeutically effective doses of anti-HER2 antibody. In this manner, a first dose of IL-2 is administered up to one month before the first dose of anti-HER2 antibody is administered. By “up to one month” is intended the first dose of IL-2 is administered at least one day before initiating anti-HER2 antibody administration, but not more than one month (i.e., 30 days) before initiating anti-HER2 antibody administration. Thus, IL-2 administration can begin, for example, 1 day, 2 days, 3 days, 4 days, 5 days, 6 days, 7 days (i.e., 1 week), 10 days, 14 days (i.e., two weeks), 17 days, 21 days (i.e., 3 weeks), 24 days, 28 days (4 weeks), or up to one month (i.e., 30 days) before administering the first therapeutically effective dose of the anti-HER2 antibody.

In other embodiments, the two-level IL-2 dosing regimen and anti-HER2 antibody administration begin concurrently on the same day, either at the same time (i.e., simultaneous administration) or at different times (i.e., sequential administration, in either order). Thus, for example, in one embodiment where concurrent therapy with these two therapeutic agents begins on day 1 of a treatment period, a first therapeutically effective dose of anti-HER2 antibody and a first dose of IL-2 would both be administered on day 1 of this treatment period. In one such embodiment, the dose of anti-HER2 antibody is administered first, followed by administration of the dose of IL-2 within about 10 minutes to about 4 hours of the completion of administering the dose of anti-HER2 antibody, such as within about 10, 15, 20, 25, 30, 45, 60, 90, 120, 150, 180, 210, or 240 minutes.

In alternative embodiments, a first therapeutically effective dose of anti-HER2 antibody is administered to the subject, for example, on day 1 of a treatment period, and the two-level IL-2 dosing regimen is initiated by administering a first dose of IL-2 within 10 days of administering the first therapeutically effective dose of anti-HER2 antibody. In such embodiments, preferably the two-level IL-2 dosing regimen is initiated by administering a first dose of IL-2 within 7 days of administering the first therapeutically effective dose of anti-HER2 antibody, such as within 1, 2, 3, 4, 5, 6, or 7 days.

Depending upon the severity of the disease, the patient's health, and prior history of the patient's disease, one or more cycles of a two-level IL-2 dosing regimen can be administered concurrently with anti-HER2 antibody therapy, where therapeutically effective doses of the antibody are administered weekly, or, alternatively, once every two, three, or four weeks.

In accordance with the methods of the present invention, a therapeutically effective dose of anti-HER2 antibody is administered weekly, or is administered once every two, three, or four weeks, in combination with one or more cycles of a constant IL-2 dosing regimen or in combination with one or more cycles of a two-level IL-2 dosing regimen. The duration of anti-HER2 antibody administration and the duration of any given cycle of either of the IL-2 dosing regimens will depend upon the subject's overall health, history of disease progression, and tolerance of the particular anti-HER2 antibody/IL-2 administration protocol. Generally, therapeutically effective doses of anti-HER2 antibody are administered weekly (i.e., once a week), or once every two-four weeks, for example, once every two weeks, once every three weeks, or once every four weeks, until the subject exhibits one or more anti-HER2 antibody toxicity symptoms, at which time dosing of the antibody, and dosing of the IL-2 if in progress, is concluded. Anti-HER2 antibody toxicity symptoms include cardiac or ventricular dysfunction, including dyspena, peripheral edema, S3 gallop, and congestive heart failure; respiratory distress; pulmonary events; and severe hypersensitivity reactions, including anaphylaxis, bronchospasm, angioedema, hypotension, and urticaria, or other indications on the package insert label of the approved anti-HER2 antibody product. The subject may resume concurrent therapy with these two therapeutic agents as needed following resolution of signs and symptoms of these anti-HER2 antibody toxicity symptoms. Resumption of concurrent therapy with these two therapeutic agents can entail either of the anti-HER2 antibody/IL-2 administration protocols disclosed herein (i.e., anti-HER2 antibody with the constant IL-2 dosing regimen or anti-HER2 antibody with the two-level IL-2 dosing regimen) depending upon the overall health of the subject, relevant disease state, and tolerance for the particular anti-HER2 antibody/IL-2 administration protocol.

The duration of IL-2 administration during concurrent therapy with these two therapeutic agents is a function of the IL-2 dosing regimen used. Generally, IL-2 is administered according to the disclosed protocols, and a subject can repeat one or more cycles of a constant or two-level IL-2 dosing regimen as needed, unless IL-2 toxicity symptoms develop. Should such toxicity symptoms develop, the subject can be taken off of IL-2 dosing until complete resolution of any observed toxicity symptoms. Such IL-2 toxicity responses include but are not limited to, chronic fatigue, nausea, hypotension, fever, chills, weight gain, pruritis or rash, dysprea, azotemia, confusion, thrombocytopenia, myocardial infarction, gastrointestinal toxicity, and vascular leak syndrome (see, for example, Allison et al. (1989) J. Clin. Oncol. 7(1):75-80).

In some embodiments of the invention, the subject undergoing concurrent therapy with these two therapeutic agents is administered one or more cycles of a constant IL-2 dosing regimen in combination with the anti-HER2 antibody dosing schedule disclosed herein (i.e., therapeutically effective doses of anti-HER2 antibody administered weekly, or administered once every two, three, or four weeks). By “constant IL-2 dosing regimen” is intended the subject undergoing concurrent therapy with IL-2 and anti-HER2 antibody is administered a constant total weekly dose of IL-2 over the course of any given cycle of IL-2 administration. One complete cycle of a constant IL-2 dosing regimen comprises administering a constant total weekly dose of IL-2 for a period of about 2 weeks to about 12 weeks, such as about 2, 3, 4, 5, 6, 7, 8, 9, 10, 11, or 12 weeks, followed by a time period off of IL-2 dosing, which has a duration of about 1 week to about 4 weeks, including 1, 2, 3, or 4 weeks. Thus each cycle of a constant IL-2 dosing regimen comprises a first time period during which the subject is administered a constant total weekly dose of IL-2, and a second time period during which IL-2 dosing is withheld. (i.e., a “rest” period or “holiday” from IL-2 administration). Preferably the subject is administered the constant total weekly dose of IL-2 for at least 4 weeks up to about 12 weeks, at which time IL-2 dosing is withheld for a period of about 1 week to about 4 weeks. During each cycle of the constant IL-2 dosing regimen, the subject remains on the recommended dosing regimen for the anti-HER2 antibody, and thus receives a therapeutically effective dose of anti-HER2 antibody according to a weekly dosing schedule, or according to a once every two weeks, once every three weeks, or once every four weeks dosing schedule.

At the discretion of the managing physician, the subject undergoing weekly anti-HER2 antibody administration, or anti-HER2 antibody administration once every two, three, or four weeks, can continue receiving the constant total weekly dose of IL-2 for an extended period of time beyond 12 weeks, for example, for an additional 1-8 weeks, providing the anti-HER2 antibody/constant IL-2 dosing protocol is well tolerated and the subject is exhibiting minimal signs of either anti-HER2 antibody and/or IL-2 toxicity symptoms. In such an embodiment, conclusion of IL-2 dosing would be followed by a period of about 1 week to about 4 weeks during which IL-2 dosing would be withheld to allow the subject time off of this therapeutic agent before starting a subsequent cycle of the constant IL-2 dosing regimen.

In one embodiment, a subject in need of concurrent therapy with anti-HER2 antibody and IL-2 is administered a therapeutically effective dose of anti-HER2 antibody once per week throughout a treatment period, or is administered a therapeutically effective dose of anti-HER2 antibody once every three weeks throughout this treatment period, beginning on day 1 of this treatment period, and the first cycle of a constant IL-2 dosing regimen is initiated beginning on day 3, 4, 5, 6, 7, 8, 9, or 10 of the same treatment period. In one such embodiment, the first cycle of the constant IL-2 dosing regimen begins on day 8 (i.e., at the start of week 2) of the treatment period, and has a duration of IL-2 administration of about 4 weeks to about 12 weeks, followed by a time period off of IL-2 administration that has a duration of about 1 week to about 4 weeks. At the conclusion of this first cycle of the constant IL-2 dosing regimen, the subject receives one or more subsequent cycles of the constant IL-2 dosing regimen as noted herein above.

In another embodiment, the subject is administered a therapeutically effective dose of anti-HER2 antibody once per week throughout a treatment period, or is administered a therapeutically effective dose of anti-HER2 antibody once every three weeks throughout this treatment period, beginning on day 1 of this treatment period, and a first cycle of the constant IL-2 dosing regimen begins on day 8 (i.e., at the start of week 2) of the treatment period, and has a duration of IL-2 administration of 4 weeks, followed by a time period off of IL-2 administration having a duration of 1 week. At the discretion of the managing physician, the subject is then administered one or more subsequent cycles of this constant IL-2 dosing regimen, i.e., administration of the constant total weekly dose of IL-2 for 4 weeks, followed by 1 week off of IL-2 administration. During the entire treatment period, the subject continues to receive the therapeutically effective dose of anti-HER2 antibody according to the once-a-week dosing schedule, or the once-every-three-weeks dosing schedule, with the proviso that the subject does not exhibit symptoms of anti-HER2 antibody toxicity. Thus, for example, if the subject undergoes three cycles of the constant IL-2 dosing regimen in combination with weekly (i.e., once-per-week) administration of anti-HER2 antibody, with the first cycle of IL-2 administration beginning on day 8 of a treatment period (i.e., day 1 of week 2), therapeutically effective doses of the anti-HER2 antibody would be administered to this subject on day 1 of each week of treatment (i.e., day 1 of weeks 1-16), and a constant total weekly dose of IL-2 would be administered during weeks 2-5, weeks 7-10, and weeks 12-15, with time off from IL-2 dosing occurring during weeks 6, 11, and 16.

In other embodiments of the invention, concurrent therapy with anti-HER antibody and IL-2 comprises administering a therapeutically effective dose of anti-HER2 antibody once per week, or once every two, three, or four weeks, throughout a treatment period in combination with one or more cycles of a “two-level IL-2 dosing regimen” during the course of this treatment period. By “two-level IL-2 dosing regimen” is intended the subject undergoing concurrent therapy with IL-2 and anti-HER2 antibody is administered IL-2 during two time periods of IL-2 dosing, which have a combined duration of about 2 weeks to about 16 weeks, including, 2, 3, 4, 5, 6, 7, 8, 9, 10, 11, 12, 13, 14, 15, or 16 weeks. In one embodiment, the two-level IL-2 dosing regimen has a combined duration of about 4 weeks to about 12 weeks; in other embodiments, the two-level IL-2 dosing regimen has a combined duration of about 4 weeks to about 8 weeks, including about 4, 5, 6, 7, or 8 weeks. The total weekly dose of IL-2 that is to be administered during the first and second time periods of the two-level IL-2 dosing regimen is chosen such that a higher total weekly dose of IL-2 is given during the first time period and a lower total weekly dose of IL-2 is given during the second time period.

The duration of the individual first and second time periods of any given cycle of the two-level IL-2 dosing regimen can vary, depending upon the health of the individual and history of disease progression. Generally, the subject is administered higher total weekly doses of IL-2 for at least 1 week out of the 4-week to 16-week two-level IL-2 dosing regimen. In one embodiment, higher total weekly doses of IL-2 are administered during the first half of the two-level IL-2 dosing regimen, with lower total weekly doses being administered during the second half of the two-level IL-2 dosing regimen. Thus, for example, where one complete cycle of the two-level IL-2 dosing regimen has a combined duration of 8 weeks, the higher total weekly doses of IL-2 would be administered for the first 4 weeks of IL-2 dosing, and the lower total weekly doses of IL-2 would be administered for the second 4 weeks of IL-2 dosing.

Though specific dosing regimens are disclosed herein below, it is recognized that the invention encompasses any administration protocol that provides for concurrent therapy with an anti-HER2 antibody and one or more cycles of a two-level IL-2 dosing regimen that provides for initial exposure to higher total weekly doses of IL-2, and subsequent exposure to lower total weekly doses of IL-2. While not being bound by theory, it is believed that administering a higher dose of IL-2 during the initial stages of IL-2 dosing provides for an initial stimulation of NK cell activity that can be maintained by a lower dose during the subsequent weeks of IL-2 dosing. As IL-2 side effects are dose-related, the lowered dose of IL-2 will increase tolerability of this therapeutic agent during the extended treatment period.

Thus, the methods of the invention contemplate treatment regimens where a therapeutically effective dose of at least one anti-HER2 antibody is administered once a week throughout a treatment period, or is administered once every two, three, or four weeks throughout this treatment period, in combination with a two-level IL-2 dosing having a combined duration of about 2 weeks to about 16 weeks, including 2, 3, 4, 5, 6, 7, 8, 9, 10, 11, 12, 13, 14, 15, or 16 weeks. Either agent could be administered first, as explained above for this two-level IL-2 dosing regimen. For example, in one embodiment, a therapeutically effective dose of anti-HER2 antibody is administered first, for example, on day 1 of a treatment period, followed by initiation of the two-level IL-2 dosing regimen within 10 days, preferably within 7 days of the first administration of the anti-HER2 antibody, for example, within 1, 2, 3, 4, 5, 6, or 7 days. During the two-level IL-2 dosing regimen, a higher total weekly dose of IL-2 is administered in the first time period of the two-level IL-2 dosing regimen, for example, over the first 1-4 weeks of IL-2 administration, and lower total weekly doses of IL-2 are administered during the second time period of the two-level IL-2 dosing regimen (i.e., over the remaining course of the two-level IL-2 dosing regimen).

In one embodiment, the methods of the invention provide for administering a therapeutically effective dose of a pharmaceutical composition comprising at least one anti-HER2 antibody weekly (i.e., once per week) throughout a treatment period, or administering this therapeutically effective dose of anti-HER-2 antibody once every three weeks throughout this treatment period, in combination with one or more cycles of a two-level IL-2 dosing regimen during the course of this treatment period, where each cycle of the two-level IL-2 dosing regimen has a combined duration of 4 weeks to 8 weeks, including 4, 5, 6, 7, or 8 weeks. Thus, for example, where the anti-HER2 antibody is to be dosed once per week, a therapeutically effective dose of at least one anti-HER2 antibody is administered on day 1 of each week of the treatment period, and the first cycle of a 4-week to 8-week two-level IL-2 dosing regimen is initiated beginning on day 3, 4, 5, 6, 7, 8, 9, or 10 of the same treatment period.

In one such embodiment, therapeutically effective doses of the pharmaceutical composition comprising the anti-HER2 antibody are administered weekly beginning on day 1 of a treatment period and continuing throughout the treatment period, and a first cycle of the two-level IL-2 dosing regimen begins on day 8 of the same treatment period and continues for 8 weeks (i.e., during weeks 2-9 of the treatment period).

The methods of the present invention also contemplate embodiments where a subject undergoing anti-HER2 antibody administration according to the dosing schedule recommended herein in combination with administration of one or more cycles of a two-level IL-2 dosing regimen is given a “drug holiday” or a time period off from TL-2 dosing between the conclusion of the first time period of any given cycle of the two-level IL-2 dosing regimen and the initiation of the second time period of that particular cycle of the two-level IL-2 dosing regimen. In these embodiments, the two-level IL-2 dosing regimen is interrupted such that IL-2 dosing is withheld for a period of about 1 week to about 4 weeks following conclusion of the first time period of a given cycle of the two-level IL-2 dosing regimen during which the higher total weekly dose has been administered. During this time period off of IL-2 dosing, the subject may continue to receive a therapeutically effective dose of anti-HER2 antibody according to the dosing schedule recommended herein (i.e., once per week, or once every two, three, or four weeks) as long as the subject does not exhibit symptoms of anti-HER antibody toxicity. The length of this interruption in IL-2 dosing will depend upon the health of the subject, history of disease progression, and responsiveness of the subject to the initial IL-2/antibody therapy received during the first time period of any given cycle of the two-level IL-2 dosing regimen. Generally, IL-2 dosing is interrupted for a period of about 1 week to about 4 weeks, at which time the subject is administered the second time period of the two-level IL-2 dosing regimen, where lower total weekly doses of IL-2 are administered in combination with the weekly administration of therapeutically effective doses of the anti-HER2 antibody. In order to complete any given cycle of a two-level IL-2 dosing regimen, a subject must be administered both the first period of higher total weekly dosing and the second period of lower total weekly dosing.

Depending upon the overall health of the subject, clinical response, and tolerability of concurrent therapy with these two therapeutic agents, following completion of a first cycle of a two-level IL-2 dosing regimen, the subject can be administered one or more subsequent cycles of a two-level IL-2 dosing regimen in combination with administration of a therapeutically effective dose of anti-HER2 antibody, where the antibody is dosed once per week or is dosed once every two, three, or four weeks. In such embodiments, the managing physician can allow for a drug holiday or time period off of IL-2 administration between successive cycles. Thus, upon completion of any given cycle of a two-level IL-2 dosing regimen, which itself may or may not include a time period off of IL-2 administration between the first and second periods of IL-2 dosing, the subject can be given a break in IL-2 administration before initiating a subsequent cycle of the two-level IL-2 dosing regimen. Generally, the time period off of IL-2 administration between any given cycle of two-level IL-2 dosing is about 1 week to about 4 weeks, including about 1, 2, 3, or 4 weeks. During IL-2 drug holidays the subject may continue to receive therapeutically effective doses of anti-HER2 antibody, which can be administered once per week, or once every two, three, or four weeks.

The need for administering multiple cycles of a constant IL-2 dosing regimen or multiple cycles of a two-level IL-2 dosing regimen is at the discretion of the managing physician and can be assessed by monitoring natural-killer (NK) cell counts in subjects undergoing treatment with the methods of the invention. In general, NK cell number should be determined by fluorescent cell sorting of CD16+ or CD56+ cells. In this manner, NK cell counts are measured bi-weekly or monthly during the constant IL-2 dosing regimen or the two-level IL-2 dosing regimen, and at the conclusion of any given cycle of IL-2 dosing before a time off (i.e., holiday) from IL-2 dosing is initiated. NK cell levels above 200 cells/μl may be an important factor influencing positive clinical response to anti-HER2 antibody therapy. Thus NK cell counts falling below this level are indicative of the need to reinstate IL-2 therapy following a time off of IL-2 dosing, for example, between cycles of constant or two-level IL-2 dosing regimens, or between the first and second periods of the two-level IL-2 dosing regimen.

For human subjects undergoing concurrent therapy with anti-HER2 antibody according to the administration schedule recommended herein in combination with one or more cycles of a constant IL-2 dosing regimen or one or more cycles of a two-level IL-2 dosing regimen, the total weekly dose of IL-2 to be administered during periods of IL-2 dosing can be administered as a single dose, or can be partitioned into a series of equivalent doses that are administered according to a two- three-, four-, five-, six-, or seven-times-a-week dosing schedule. Where higher total weekly doses are to be administered during a first time period, and lower total weekly doses are to be administered during a second time period, it is not necessary that the total weekly dose be administered in the same manner over the course of both dosing periods. Thus, for example, the higher total weekly dose during the first time period of a two-level IL-2 dosing regimen can be administered as a single dose, or can be partitioned into a series of equivalent doses that are administered according to a two- three-, four-, five-, six-, or seven-times-a-week dosing schedule. Similarly, the lower total weekly dose during the second time period of a two-level IL-2 dosing regimen can be administered as a single dose, or can be partitioned into a series of equivalent doses that are administered according to a two-, three-, four-, five-, six-, or seven-times-a-week dosing schedule.

For purposes of the present invention, a “two-, three-, four-, five-, six-, or seven-times-a-week dosing schedule” is intended to mean that the total weekly dose is partitioned into two, three, four, five, six, or seven equivalent doses, respectively, which are administered to the subject over the course of a 7-day period, with no more than one equivalent dose being administered per 24-hour period. The series of equivalent doses can be administered on sequential days, or can be administered such that one or more days occur between any two consecutive doses, depending upon the total number of equivalent doses administered per week.

Thus, for example, where a series of two equivalent doses of IL-2 are administered per week (i.e., over a 7-day period) and the first equivalent dose of that week is administered on day 1, the second equivalent dose of IL-2 can be administered on day 2, 3, 4, 5, 6, or 7 of that week. In one embodiment, the total weekly dose of IL-2 is partitioned into two equivalent doses that are administered to the subject within a 7-day period, allowing for a minimum of 72 hours between doses and a maximum of 96 hours between doses.

Similarly, where a series of three equivalent doses of IL-2 are administered per week and the first equivalent dose of that week is administered on day 1, the second equivalent dose can be administered on day 2, 3, 4, 5, or 6 of that week, and the third equivalent dose can be administered on day 3, 4, 5, 6, or 7 of that week, so long as about 24 hours occur between administration of the second and third equivalent doses. In one embodiment, the total weekly dose of IL-2 is partitioned into three equivalent doses that are administered to the subject within a 7-day period, allowing for a minimum of 25 hours between doses and a maximum of 72 hours between doses.

Where a series of four equivalent doses of IL-2 are administered per week and the first equivalent dose of that week is administered on day 1, the second equivalent dose can be administered on day 2, 3, 4, or 5 of that week, the third equivalent dose can be administered on day 3, 4, 5, or 6 of that week, and the fourth equivalent dose can be administered on day 4, 5, 6, or 7 of that week, so long as about 24 hours occur between administration of any two consecutive doses (i.e., between the first and second equivalent doses, between the second and third equivalent doses, and between the third and fourth equivalent doses).

Where a series of five equivalent doses are administered per week and the first equivalent dose of that week is administered on day 1, the second equivalent dose can be administered on day 2, 3, or 4 of that week, the third equivalent dose can be administered on day 3, 4, or 5 of that week, the fourth equivalent dose can be administered on day 4, 5, or 6 of that week, and the fifth equivalent dose can be administered on day 5, 6, or 7 of that week, so long as about 24 hours occur between administration of any two consecutive doses (i.e., between the first and second equivalent doses, between the second and third equivalent doses, between the third and fourth equivalent doses, and between the fourth and fifth equivalent doses).

Where a series of six equivalent doses of IL-2 are administered per week and the first equivalent dose of that week is administered on day 1, the second equivalent dose can be administered on day 2 or 3 of that week, the third equivalent dose can be administered on day 3 or 4 of that week, the fourth equivalent dose can be administered on day 4 or 5 of that week, the fifth equivalent dose can be administered on day 5 or 6 of that week, and the sixth equivalent dose can be administered on day 6 or 7 of that week, so long as about 24 hours occur between administration of any two consecutive doses (i.e., between the first and second equivalent doses, between the second and third equivalent doses, between the third and fourth equivalent doses, between the fourth and fifth equivalent doses, and between the fifth and sixth equivalent doses).

In one embodiment, the total weekly dose of IL-2 is partitioned into seven equivalent doses, which are administered daily over the 7-day period, with about 24 hours occurring between each consecutive dose.

It is not necessary that the same dosing schedule be followed throughout a constant IL-2 dosing regimen, or that the same dosing schedule be followed for both the first and second periods of the two-level IL-2 dosing regimen. Thus, the dosing schedule can be adjusted to accommodate an individual's tolerance of prolonged IL-2 therapy in combination with anti-HER2 antibody therapy, and to reflect the individual's responsiveness to concurrent therapy with these two therapeutic agents. The preferred dosing schedule during the constant IL-2 dosing regimen and the two time periods of the two-level IL-2 dosing regimen is readily determined by the managing physician given the patient's medical history and the guidance provided herein.

Thus, the present invention provides methods for treating a human subject with a cancer characterized by overexpression of the HER2 receptor protein using concurrent therapy with anti-HER2 antibody administered according to the dosing schedule recommended herein in combination with one or more cycles of either a constant IL-2 dosing regimen or a two-level IL-2 dosing regimen. For purposes of the present invention, the therapeutically effective dose of anti-HER2 antibody to be administered on a weekly schedule, or once every two, three, or four weeks, concurrent with one or more cycles of a constant IL-2 or two-level IL-2 dosing regimen ranges from about 1.0 mg/kg body weight to about 10.0 mg/kg body weight. In some embodiments, the therapeutically effective dose of anti-HER2 antibody is about 1.0 mg/kg to about 8.0 mg/kg, about 1.0 mg/kg to about 6.0 mg/kg, or about 1.0 mg/kg to about 4.0 mg/kg, including 1.0 mg/kg, 1.5 mg/kg, 2.0 mg/kg, 2.5 mg/kg, 3.0 mg/kg, 3.5 mg/kg, 4.0 mg/kg, and other such values within this range. The same therapeutically effective dose of anti-HER2 antibody can be administered each week of dosing. Alternatively, different therapeutically effective doses of anti-HER2 antibody can be used over the course of a treatment period. Thus, in some embodiments, the initial therapeutically effective dose of anti-HER2 antibody can be in the higher dosing range (i.e., about 3.5 mg/kg body weight to about 10.0 kg/mg body weight), with subsequent doses falling within the lower dosing range (i.e., about 1.0 mg/kg body weight to about 3.5 mg/kg body weight). In one embodiment, the initial therapeutically effective dose of anti-HER2 antibody is about 3.5 mg/kg body weight to about 5.0 mg/kg body weight, including about 3.5 mg/kg, about 4.0 mg/kg, about 4.5 mg/kg, and about 5.0 mg/kg body weight, and subsequent therapeutically effective doses of anti-HER2 antibody are about 1.5 mg/kg to about 3.0 mg/kg body weight, including about 1.5 mg/kg, about 2.0 mg/kg, about 2.5 mg/kg, and about 3.0 mg/kg. In a preferred embodiment, the initial therapeutically effective dose of anti-HER2 antibody is about 4.0 mg/kg body weight, and subsequent therapeutically effective doses of anti-HER2 antibody are about 2.0 mg/kg body weight. The pharmaceutical composition comprising the anti-HER2 antibody is administered, for example, intravenously, as noted herein above.

In accordance with the methods of the present invention, the IL-2 is administered, for example, by IV, IM, or SC injection, in combination with the anti-HER2 antibody therapy so as to provide the recommended total weekly doses of IL-2 during the constant IL-2 dosing regimen or during the two-level IL-2 dosing regimen as described more fully below. The following embodiments provide guidance as to suitable total weekly doses and dosing regimens for IL-2, though any number of different dosing regimens can be contemplated by one of skill in the art once apprised of the disclosure set forth herein.

For purposes of the following discussion of therapeutically effective doses of IL-2, the monomeric IL-2 pharmaceutical formulation referred to herein as “L2-7001 IL-2” is used as the reference IL-2 standard. By “reference IL-2 standard” is intended the formulation of IL-2 that serves as the basis for determination of the therapeutically effective doses to be administered to a subject with a cancer characterized by overexpression of the HER2 receptor protein in combination with at least one anti-HER2 antibody to achieve the desired positive effect, i.e., a positive therapeutic response that is improved with respect to that observed with either of these anti-tumor agents alone. This liquid formulation comprises the same human IL-2 mutein (aldesleukin) as Proleukin® IL-2 (available commercially from Chiron Corporation, Emeryville, Calif.) with the exception of the final purification steps prior to its formulation as L2-7001 according to the method disclosed in the copending application entitled “Stabilized Liquid Polypeptide-Containing Pharmaceutical Compositions,” filed Oct. 3, 2000, and assigned U.S. application Ser. No. 09/677,643. See Example 2 below. The therapeutically effective dose of L2-7001 IL-2 to be administered to a subject in need of concurrent therapy with anti-HER2 antibody and IL-2 depends upon the medical condition of the subject, and the recommended dosing regimen (i.e., constant versus two-level) as noted above.

In accordance with the methods of the present invention, the total weekly dose of L2-7001 IL-2 to be administered concurrently with weekly administration of a therapeutically effective dose of anti-HER2 antibody as defined above can be about 270 μg to about 1620 μg, depending upon the medical history of the patient undergoing therapy and the recommended IL-2 dosing regimen (i.e., constant versus two-level IL-2 dosing regimen). Where higher doses of IL-2 are to be administered, the total weekly dose of L2-7001 can be in the range from about 840 μg to about 1620 μg, including about 840 μg, 900 μg, 960 μg, 1020 μg, 1080 μg, 1140 μg, 1200 μg, 1260 μg, 1350 μg, 1500 μg, and 1620 μg, and other such values falling within this range. Where intermediate doses of IL-2 are to be administered, the total weekly dose of L2-7001 IL-2 can be in the range from about 600 μg to about 840 μg, including about 600 μg, 630 μg, 660 μg, 690 μg, 720 μg, 750 μg, 780 μg, 810 μg, 840 μg, and other such values falling within this range. Where lower doses of IL-2 are to be administered, the total weekly dose of L2-7001 IL-2 can be in the range of about 270 μg to about 600 μg, including about 270 μg, 300 μg, 330 μg, 360 μg, 390 μg, 420 μg, 450 μg, 480 μg, 510 μg, 540 μg, 570 μg, 600 μg, and other such values falling within this range. These total weekly doses represent absolute doses. The corresponding relative doses are readily calculated. The average person is approximately 1.7 m². Thus, for example, where the absolute total weekly dose of L2-7001 IL-2 to be administered is about 270 μg to about 600 μg (i.e., within the lower dosing range), the relative total weekly dose of L2-7001 to be administered is about 159 μg/m² to about 353 μg/m².

Where L2-7001 IL-2 is to be administered according to a constant IL-2 dosing regimen, the total weekly dose is about 270 μg up to about 1620 μg, preferably about 600 μg to about 1350 μg. Thus, for example, in some embodiments, the total amount of L2-7001 IL-2 that is to be administered per week as part of a constant IL-2 dosing regimen is about 270 μg, 360 μg, 450 μg, 600 μg, 660 μg, 720 μg, 780 μg, 810 μg, 840 μg, 900 μg, 960 μg, 1080 μg, 1200 μg, 1350 μg, 1440 μg, 1500 μg, 1560 μg, 1620 μg, or other such values falling within the range of about 270 μg to about 1620 μg. In one embodiment, the total weekly dose of L2-7001 IL-2 is about 840 μg to about 1080 μg. In other embodiments, the total weekly dose of L2-7001 is about 600 μg to about 840 μg.

As previously noted, the total weekly dose of IL-2 during a constant IL-2 dosing regimen can be administered as a single dose, or can be partitioned into a series of equivalent doses that are administered according to a two-, three-, four-, five-, six- or seven-times-a-week dosing schedule. Thus, for example, where the total weekly dose of the reference IL-2 standard L2-7001 IL-2 is 270 μg, the three equivalent doses of this reference IL-2 standard to be administered during each week would be 90 μg, and the two equivalent doses of this reference IL-2 standard to be administered during each week would be 135 μg. Similarly, where the total weekly dose of L2-7001 IL-2 is 1620 μg, the three equivalent doses of this reference IL-2 standard to be administered during each week of IL-2 dosing would be 540 μg, and the two equivalent doses of this reference IL-2 standard to be administered during each week of IL-2 dosing would be 810 μg.

Where L2-7001 IL-2 is to be administered according to a two-level IL-2 dosing regimen, the higher total weekly dose that is administered during the first time period of this dosing regimen is about 600 μg to about 1620 μg, and the lower total weekly dose that is administered during the second time period of this dosing regimen is about 360 μg to about 1080 μg. As previously noted, the total weekly dose administered during the first time period of the two-level IL-2 dosing regimen, for example, during the first half of this dosing regimen, is always higher than the total weekly dose administered during the second time period of the two-level IL-2 dosing regimen, for example, during the second half of this dosing regimen.

Thus, in some embodiments, the higher total weekly dose of L2-7001 IL-2 that is administered during the first time period of any given cycle of the two-level IL-2 dosing regimen is about 600 μg to about 1080 μg, including about 600 μg, 660 μg, 720 μg, 780 μg, 840 μg, 900 μg, 960 μg, 1020 μg, 1080 μg, and other such values falling within this higher dosing range; and the lower total weekly dose of L2-7001 IL-2 is about 360 μg to about 840 μg, including about 360 μg, 390 μg, 420 μg, 450 μg, 480 μg, 510 μg, 540 μg, 570 μg, 600 μg, 630 μg, 660 μg, 690 μg, 720 μg, 750 μg, 780 μg, 810 μg, 840 μg, and other such values falling within this lower dosing range. In one embodiment, the two-level IL-2 dosing regimen has a combined duration of 4 weeks to 8 weeks, where the higher total weekly dose of L2-7001 IL-2 that is administered during the first time period of the two-level IL-2 dosing regimen is about 600 μg to about 840 μg, such as 600 μg, 630 μg, 660 μg, 690 μg, 720 μg, 750 μg, 780 μg, 810 μg, or 840 μg, and the lower total weekly dose of L2-7001 IL-2 that is administered during the second time period of the two-level IL-2 dosing regimen is about 360 μg to about 600 μg, such as 360 μg, 390 μg, 420 μg, 450 μg, 480 μg, 510 μg, 540 μg, or 570 μg, or 588 μg. In one such embodiment, the higher total weekly dose of L2-7001 IL-2 that is administered during the first time period is about 840 μg, and the lower total weekly dose of L2-7001 IL-2 that is administered during the second time period is about 600 μg. In another embodiment, the higher total weekly dose of L2-7001 IL-2 that is administered during the first time period is about 1080 μg, and the lower total weekly dose of L2-7001 IL-2 that is administered during the second time period is about 840 μg.

As previously noted, the total weekly dose of IL-2 during the first and second time periods of a two-level IL-2 dosing regimen is administered as a single dose, or is partitioned into a series of equivalent doses that are administered according to a two-, three-, four-, five-, six-, or seven-times-a-week dosing schedule. Thus, for example, where the total weekly dose of L2-7001 IL-2 during the first period of the two-level IL-2 dosing regimen is about 840 μg, the three equivalent doses of this reference IL-2 standard to be administered during each week would be about 280 μg, and the two equivalent doses of this reference IL-2 standard to be administered during each week would be about 420 μg. Similarly, where the total weekly dose of L2-7001 IL-2 during the second period of the two-level IL-2 dosing regimen is about 600 μg, the three equivalent doses of this reference IL-2 standard to be administered during each week would be about 200 μg, and the two equivalent doses of this reference IL-2 standard to be administered during each week would be about 300 μg.

In a preferred embodiment, the therapeutically effective dose of anti-HER2 antibody is administered according to a weekly dosing schedule beginning on day 1 of a treatment period, and the first cycle of two-level IL-2 dosing regimen is initiated on day 8 of this treatment period and has a combined duration of 8 weeks. In this embodiment, the higher total weekly dose of IL-2 administered during weeks 2-5 of the treatment period is about 600 μg to about 1080 μg, preferably about 600 μg to about 840 μg, and the lower total weekly dose of IL-2 administered during weeks 6-9 is about 360 μg to about 840 μg, preferably about 360 μg to about 600 μg. The higher and lower total weekly doses of IL-2 are administered as a single dose, or are partitioned into equivalent doses that are administered according to a two-, three-, four-, five-, six-, or seven-times-a-week dosing schedule. In one such embodiment, the higher total weekly dose of IL-2 during weeks 2-5 of the treatment period is about 600 μg to about 840 μg, for example, 840 μg, and the lower total weekly dose or IL-2 is about 360 μg to about 600 μg, for example, 600 μg. In this embodiment, each of the higher and lower total weekly doses of IL-2 are partitioned into two equivalent doses that are administered according to a two-times-a-week dosing schedule, where the two equivalent doses are administered to the subject within a 7-day period, allowing for a minimum of 72 hours between doses and a maximum of 96 hours between doses. In an alternative embodiment, each of the higher and lower total weekly doses of IL-2 are partitioned into three equivalent doses that are administered according to a three-times-a-week dosing schedule, where the three equivalent doses are administered to the subject within a 7-day period, allowing for a minimum of 25 hours between doses and a maximum of 72 hours between doses.

For purposes of describing this invention, the doses of IL-2 have been presented using L2-7001 IL-2 as the reference IL-2 standard. One of skill in the art can readily determine what the corresponding doses would be for any IL-2 product comprising any form of IL-2 using a conversion factor based on comparative pharmacokinetic (PK) data and the serum concentration-time curve (AUC) for PK data collected during a 24-hour period for L2-7001 IL-2. Using PK data, the IL-2 exposure in human subjects that are administered a single dose of the reference IL-2 standard is determined. These subjects are selected such that they have not previously received exogenous IL-2 therapy (i.e., these subjects are naive to IL-2 therapy). By “exogenous IL-2 therapy” is intended any intervention whereby a subject has been exposed to an exogenous source of IL-2, as opposed to exposure that occurs with the body's production of naturally occurring IL-2. Some of these subjects receive a single dose of 50 μg of the reference IL-2 standard, while others receive a single dose of 90, 135, or 180 μg of the reference IL-2 standard. See Example 4 herein below.

Following administration of the single dose of the reference IL-2 standard L2-7001, the IL-2 exposure in the blood serum is monitored over the first 10 to 12 hours post-injection, then extrapolated to 24 hours, and the resulting area under the serum concentration-time curve (AUC) for data collected during that 24-hour period is calculated. This area under the serum concentration-time curve is referred to herein as the AUC₀₋₂₄. Methods for measuring IL-2 exposure in this manner are well known in the art. See, for example, Gustavson (1998) J. Biol. Response Modifiers 1998:440-449; Thompson et al. (1987) Cancer Research 47:4202-4207; Kirchner et al. (1998) Br. J. Clin. Pharmacol. 46:5-10; Piscitelli et al. (1996) Pharmacotherapy 16(5):754-759; and Example 4 below. Thus, for those subjects receiving a dose of 50 μg L2-7001 IL-2, the AUC₀₋₂₄ value was 60 IU·hr/ml (SD=11); for those subjects receiving a dose of 90 μg of L2-7001 IL-2, the AUC₀₋₂₄ value was 110 IU·hr/ml (SD=36); for those subjects receiving a dose of 135 μg of L2-7001 IL-2, the AUC₀₋₂₄ value was 143 IU·hr/ml (SD=41); and for those subjects receiving the 180 μg dose of L2-7001 IL-2, the AUC₀₋₂₄ value was 275 IU·hr/ml (SD=99) (see Example 4 below).

The sum of individual AUC₀₋₂₄ from individual doses will comprise the total weekly AUC₀₋₂₄ in partitioned individual doses. For example, if a dose of 90 μg is administered three-times-a-week, the individual AUC₀₋₂₄ is estimated at 110 IU·hr/ml, and the total weekly AUC₀₋₂₄ will be 330 IU·hr/ml based on linear assumption of increased AUC₀₋₂₄ with dose as shown in the Table 1 below.

TABLE 1
AUC_{0-24 values} obtained after administration of L2-7001 IL-2.

| L2-7001 IL-2 Dose | AUC₀₋₂₄ |
|---|---|
| (μg) | (IU*hr/ml) |
| 90 | 110 |
| 150 | 183 |
| 210 | 257 |
| 270 | 330 |

The same total weekly AUC₀₋₂₄ of 330 IU·hr/ml could also be obtained by dosing two-times-a-week at 135 μg or dosing five-times-a-week at about 54 μg.

For any other source of IL-2 (i.e., any other IL-2 formulation or any form of IL-2, including native or biologically active variant thereof such as muteins), a comparable recommended dose for use in the methods of the invention can be determined based on this AUC₀₋₂₄ data for L2-7001 IL-2. In this manner, a single dose of the IL-2 source of interest is administered to a human subject, and the level of IL-2 in the serum following this initial IL-2 exposure is determined by collecting PK data and generating an AUC₀₋₂₄ for the IL-2 source of interest. By “initial IL-2 exposure” is intended the subject used to measure IL-2 exposure has not previously undergone therapy with an exogenous source of IL-2 as noted above. This AUC₀₋₂₄ is then compared to the AUC₀₋₂₄ for L2-7001 IL-2 to determine a conversion factor that can be used to calculate a dose of the IL-2 source that is comparable to the recommended dose for L2-7001 IL-2. See, for example, the calculations for a representative multimeric IL-2 formulation, Proleukin® IL-2, that are shown in Example 4 below. Thus, for any IL-2 source used in the methods of the present invention, the total weekly dose of IL-2 to be administered during any given cycle of a constant IL-2 dosing regimen, or during any given cycle of a two-level IL-2 dosing regimen, is in an amount equivalent to the recommended total weekly dose of the reference IL-2 standard, i.e., L2-7001 IL-2, as determined by the area under the serum concentration-time curve from human PK data.

Thus, for example, where the source of the IL-2 is Proleukin® IL-2, the total weekly dose of Proleukin® to be administered concurrently with weekly administration of a therapeutically effective dose of anti-HER2 antibody is in the range from about 18 MIU to about 54 MIU, depending upon the medical history of the patient undergoing therapy and the recommended IL-2 dosing regimen (i.e., constant versus two-level IL-2 dosing regimen). Where higher doses of IL-2 are to be administered, the total weekly dose of Proleukin® IL-2 can be in the range from about 42.0 MIU to about 54.0 MIU, including about 42.0 MIU, 43.5 MIU, 45.0 MIU, 46.5 MIU, 48.0 MIU, 49.5 MIU, 51.0 MIU, 52.5 MIU, and 54.0 MIU, and other such values falling within this range. Where intermediate doses of IL-2 are to be administered, the total weekly dose of Proleukin® IL-2 can be in the range from about 30.0 MIU to about 42.0 MIU, including about 30.0 MIU, 31.5 MIU, 33.0 MIU, 34.5 MIU, 36.0 MIU, 37.5 MIU, 39.0 MIU, 40.5 MIU, and 42.0 MIU, and other such values falling within this range. Where lower doses of IL-2 are to be administered, the total weekly dose of Proleukin® IL-2 can be in the range from about 18.0 MIU to about 30.0 MIU, including about 18.0 MIU, 19.5 MIU, 21.0 MIU, 22.5 MIU, 24.0 MIU, 25.5 MIU, 27.0 MIU, 28.5 MIU, and 30.0 MIU, and other such values falling within this range.

The foregoing total weekly doses of Proleukin® IL-2 are expressed in terms of MIU, which represent total amounts or absolute doses that are to be administered to a human subject on a weekly basis. The corresponding relative total weekly dose of Proleukin® IL-2 to be administered to a person to can readily be calculated. The average person is approximately 1.7 m². Thus, where the absolute total weekly dose of Proleukin®) IL-2 to be administered is about 42.0 MIU to about 54.0 MIU, the corresponding relative total weekly dose of Proleukin® IL-2 is about 24.7 MIU/m² to about 31.8 MIU/m². Similarly, when the absolute total weekly dose is about 30.0 MIU to 42.0 MIU, the corresponding relative total weekly dose is about 17.6 MIU/m² to about 24.7 MIU/m². When the absolute total weekly dose is about 18.0 MIU to about 30.0 MIU, the corresponding relative total weekly dose is about 10.6 MIU/m² to about 17.6 MIU/m².

MIU represents an international unit for a protein's biological activity. The international unit for IL-2 biological activity was established in 1988 by the World Health Organization (WHO) International Laboratory for Biological Standards. The IL-2 biological reference materials provided by the National Institute for Biological Standards and Control (NIBSC), which belongs to WHO, has 100 international units per ampoule of native human, Jurkat-derived IL-2. Activity of an IL-2 product can be measured against this international standard in an in vitro potency assay by HT-2 cell proliferation. Thus, for example, Proleukin® IL-2 has a biological activity of about 16.36 MIU per mg of this IL-2 product as determined by an HT-2 cell proliferation assay (see, for example, Gearing and Thorpe (1988) J. Immunological Methods 114:3-9; Nakanishi et al. (1984) J. Exp. Med. 160(6):1605-1621). The active moiety used in this product is the recombinant human IL-2 mutein aldesleukin (referred to as des-alanyl-1, serine-125 human interleukin-2; see U.S. Pat. No. 4,931,543, herein incorporated by reference). Using this information, one can calculate the recommended therapeutically effective absolute dose of Proleukin® IL-2 in micrograms.

Hence, where the absolute total weekly dose of Proleukin® is about 18.0 MIU to about 54.0 MIU, the corresponding absolute total weekly dose of Proleukin® IL-2 in micrograms is about 1100 μg to about 3300 μg of this product. Similarly, where the absolute total weekly dose in MIU is about 18.0 MIU to about 30.0 MIU, the corresponding absolute total weekly dose of Proleukin® IL-2 in μg is about 1100 μg to about 1833 μg. Where the absolute total weekly dose of Proleukin® IL-2 in MIU is about 30.0 MIU to about 42.0 MIU, the corresponding absolute total weekly dose in μg is about 1833 μg to about 2567 μg. Thus, given an absolute total weekly dose of Proleukin® IL-2 expressed in MIU, one of skill in the art can readily compute the corresponding absolute total weekly dose expressed in either MIU/m² or μg of this particular IL-2 product.

Where Proleukin® IL-2 is to be administered according to a constant IL-2 dosing regimen, the total weekly dose is about 18.0 MIU to about 54.0 MIU, preferably about 18.0 MIU to about 42.0 MIU. Thus, for example, in some embodiments, the total amount of Proleukin® IL-2 that is to be administered per week as part of a constant IL-2 dosing regimen is about 18.0 MIU, 22.5 MIU, 30.0 MIU, 33.0 MIU, 36.0 MIU, 39.0 MIU, or 42.0 MIU. In one embodiment, the total weekly dose of Proleukin® IL-2 is about 30.0 MIU to about 42.0 MIU. In other embodiments, the total weekly dose of Proleukin® IL-2 is about 18.0 MIU to about 30.0 MIU.

As previously noted, the total weekly dose of IL-2 during a constant IL-2 dosing regimen can be administered as a single dose, or can be partitioned into a series of equivalent doses that are administered according to a two-, three-, four-, five-, six- or seven-times-a-week dosing schedule. Thus, for example, where the total weekly dose of Proleukin® IL-2 is 42.0 MIU, the three equivalent doses of this IL-2 source to be administered during each week would be 14.0 MIU, and the two equivalent doses of this IL-2 source to be administered during each week would be 21.0 MIU. Similarly, where the total weekly dose of Proleukin® IL-2 is 30.0 MIU, the three equivalent doses of this IL-2 source to be administered during each week of IL-2 dosing would be 10.0 MIU, and the two equivalent doses of this IL-2 source to be administered during each week of IL-2 dosing would be 15 MIU.

Where two-level IL-2 dosing is contemplated in the methods of the present invention, the corresponding dose of Proleukin® IL-2 is as follows. An intermediate or higher dose of Proleukin® IL-2 (i.e., about 10.0 MIU to about 18.0 MIU, preferably about 10.0 MIU to about 14.0 MIU) is administered during the first period of the two-level IL-2 dosing regimen, such as over the first 2-6 weeks of IL-2 administration, followed by a shift toward administering doses in the lower to intermediate IL-2 dosing range (i.e., about 6.0 MIU to about 14.0 MIU, preferably about 6.0 MIU to about 10.0 MIU Proleukin® IL-2) throughout the remainder of the treatment period.

Thus, for example, where the subject undergoes concurrent therapy with weekly administration of anti-HER2 antibody and a two level-IL-2 dosing regimen having a combined duration of 8 weeks beginning on day 1 of week 2 (i.e., on day 8 following the first dosing with anti-HER2 antibody), the absolute total weekly dose of Proleukin® IL-2 to be administered during weeks 2-5 of the treatment period is in the range of about 30.0 MIU to about 54.0 MIU, such as 30.0 MIU, 33.0 MIU, 36.0 MIU, 39.0 MIU, 42.0 MIU, 45.0 MIU, 48.0 MIU, 51.0 MIU, or 54.0 MIU, or other such values falling within this range. In this embodiment, the absolute total weekly dose of Proleukin® IL-2 to be administered during weeks 6-9 of the treatment period is in the range of about 18.0 MIU to about 42.0 MIU or about 18.0 MIU to about 36.0 MIU, such as about 18.0 MIU, 21.0 MIU, 24.0 MIU, 27.0 MIU, 30.0 MIU, 33.0 MIU, or 36.0 MIU, and other such values falling within this range. As previously noted, the absolute total weekly dose to be administered during the first and second periods of the two-level IL-2 dosing regimen are chosen from within these ranges such that a higher absolute total weekly dose is administered during the first period of the two-level IL-2 dosing regimen (for example, weeks 2-5 of a 9-week treatment period), and a lower absolute total weekly dose is administered during the second period of the two-level IL-2 dosing regimen (for example, weeks 6-9 of the same 9-week treatment period). Thus, for example, where the absolute total weekly dose of IL-2 during the first period of the two-level IL-2 dosing regimen is about 48.0 MIU to about 54.0 MIU, the absolute total weekly dose of Proleukin® IL-2 during the second period of IL-2 dosing preferably falls within the range of about 18.0 MIU to about 42.0 MIU.

Thus, where the duration of a treatment period with concurrent therapy with weekly anti-HER2 antibody dosing and a two-level IL-2 dosing regimen is about 9 weeks, in one embodiment the absolute total weekly dose of Proleukin® IL-2 during weeks 2-5 of the treatment period is about 30.0 MIU to about 54.0 MIU or about 30.0 MIU to about 42.0 MIU, and the absolute total weekly dose of Proleukin® IL-2 during weeks 6-9 of this treatment period is about 18.0 MIU to about 30.0 MIU. In this embodiment, the corresponding relative total weekly doses of Proleukin® IL-2 during weeks 2-5 are about 17.6 MIU/m to about 31.8 MIU/m² or about 17.6 MIU/m² to about 24.7 MIU/m², respectively. The corresponding relative total weekly doses Proleukin® IL-2 during weeks 6-9 are about 10.6 MIU/m² to about 17.6 MIU/m². For this same embodiment, the corresponding absolute total weekly doses of Proleukin® IL-2 expressed in μg for weeks 2-5 is about 1833 μg to about 3300 μg or about 1833 μg to about 2566 μg, respectively. The corresponding absolute total weekly dose of Proleukin® IL-2 expressed in μg for weeks 6-9 is about 1100 μg to about 1833 μg.

In one embodiment, the absolute total weekly dose of Proleukin® IL-2 during weeks 2-5 is about 42.0 MIU, and the absolute total weekly dose of Proleukin® IL-2 during weeks 6-9 is about 30.0 MIU. In this embodiment, each of the higher and lower total weekly doses of Proleukin® IL-2 are partitioned into two equivalent doses that are administered according to a two-times-a-week dosing schedule, where the two equivalent doses are administered to the subject within a 7-day period, allowing for a minimum of 72 hours between doses and a maximum of 96 hours between doses. In an alternative embodiment, each of the higher and lower total weekly doses of Proleukin® IL-2 are partitioned into three equivalent doses that are administered according to a three-times-a-week dosing schedule, where the three equivalent doses are administered to the subject within a 7-day period, allowing for a minimum of 25 hours between doses and a maximum of 72 hours between doses.

Generally, a subject undergoing therapy in accordance with the previously mentioned dosing regimens remains on the particular dosing regimen until the subject exhibits one or more anti-HER2 antibody toxicity symptoms, at which time dosing of both of these agents is concluded. If IL-2 toxicity symptoms develop, IL-2 dosing can be withdrawn. The subject may resume concurrent therapy as needed following resolution of signs and symptoms of anti-HER2 antibody or IL-2 toxicity symptoms.

The administration protocols of the present invention provide an improved means for managing cancers that are characterized by overexpression of the HER2 receptor protein in a human patient. The constant IL-2 dosing schedule with interruptions between provides an intermittent dosing schedule that allows for less frequent administration of the IL-2 during anti-HER2 antibody therapy, and better tolerability of long-term IL-2 therapy. The two-level IL-2 dosing regimen offers the opportunity to provide a patient with higher total weekly doses of IL-2, which provide for expansion of NK cell numbers that can be maintained by a lower dose during the subsequent weeks of IL-2 dosing. As IL-2 side effects are dose-related, the lowered dose will increase tolerability during the extended treatment period. This administration protocol has the additional attraction of providing IL-2 drug holidays between the higher and lower total weekly IL-2 dosing schedules, as well as IL-2 drug holidays between completed cycles of the two-level IL-2 dosing regimen, again contributing to increased tolerability of concurrent therapy with anti-HER2 antibody and IL-2.

The term “IL-2” as used herein refers to a lymphokine that is produced by normal peripheral blood lymphocytes and is present in the body at low concentrations. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J. Exp. Med. 159:1709) and has an isoelectric point in the range of 6-8.5. For purposes of the present invention, the term “IL-2” is intended to encompass any source of IL-2, including mammalian sources such as, e.g., mouse, rat, rabbit, primate, pig, and human, and may be native or obtained by recombinant techniques, such as recombinant IL-2 polypeptides produced by microbial hosts. The IL-2 may be the native polypeptide sequence, or can be a variant of the native IL-2 polypeptide as described herein below, so long as the variant IL-2 polypeptide retains the IL-2 biological activity of interest as defined herein. Preferably the IL-2 polypeptide or variant thereof is derived from a human source, and includes human IL-2 that is recombinantly produced, such as recombinant human IL-2 polypeptides produced by microbial hosts, and variants thereof that retain the IL-2 biological activity of interest. Any pharmaceutical composition comprising IL-2 as a therapeutically active component can be used to practice the present invention.

The pharmaceutical compositions useful in the methods of the invention may comprise biologically active variants of IL-2. Such variants should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein, including assays described in the present invention. Additionally, antibodies raised against a biologically active native polypeptide can be tested for their ability to bind to the variant polypeptide, where effective binding is indicative of a polypeptide having a conformation similar to that of the native polypeptide.

For purposes of the present invention, the IL-2 biological activity of interest is the ability of IL-2 to activate and/or expand natural killer (NK) cells to mediate lymphokine activated killer (LAK) activity and antibody-dependent cellular cytotoxicity (ADCC). Thus, an IL-2 variant (for example, a mutein of human IL-2) for use in the methods of the present invention will activate and/or expand NK cells to mediate LAK activity and ADCC. Assays to determine IL-2 activation or expansion of NK cells and mediation of LAC or ADCC activity are well known in the art.

Suitable biologically active variants of native or naturally occurring IL-2 can be fragments, analogues, and derivatives of that polypeptide. By “fragment” is intended a polypeptide consisting of only a part of the intact polypeptide sequence and structure, and can be a C-terminal deletion or N-terminal deletion of the native polypeptide. By “analogue” is intended an analogue of either the native polypeptide or of a fragment of the native polypeptide, where the analogue comprises a native polypeptide sequence and structure having one or more amino acid substitutions, insertions, or deletions. “Muteins”, such as those described herein, and peptides having one or more peptoids (peptide mimics) are also encompassed by the term analogue (see International Publication No. WO 91/04282). By “derivative” is intended any suitable modification of the native polypeptide of interest, of a fragment of the native polypeptide, or of their respective analogues, such as glycosylation, phosphorylation, polymer conjugation (such as with polyethylene glycol), or other addition of foreign moieties, so long as the desired biological activity of the native polypeptide is retained. Methods for making polypeptide fragments, analogues, and derivatives are generally available in the art.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor Laboratory Press, Plainview, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of the IL-2 polypeptide of interest, modifications are made such that variants continue to possess the desired activity. Obviously, any mutations made in the DNA encoding the variant polypeptide must not place the sequence out of reading frame and preferably will not create complementary regions that could produce secondary mRNA structure. See EP Patent Application Publication No. 75,444.

Biologically active variants of IL-2 will generally have at least 70%, preferably at least 80%, more preferably about 90% to 95% or more, and most preferably about 98% or more amino acid sequence identity to the amino acid sequence of the reference polypeptide molecule, which serves as the basis for comparison. Thus, where the IL-2 reference molecule is human IL-2, a biologically active variant thereof will have at least 70%, preferably at least 80%, more preferably about 90% to 95% or more, and most preferably about 98% or more sequence identity to the amino acid sequence for human IL-2. A biologically active variant of a native polypeptide of interest may differ from the native polypeptide by as few as 1-15 amino acids, as few as 1-10, such as 6-10, as few as 5, as few as 4, 3, 2, or even 1 amino acid residue. By “sequence identity” is intended the same amino acid residues are found within the variant polypeptide and the polypeptide molecule that serves as a reference when a specified, contiguous segment of the amino acid sequence of the variants is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

Thus, the determination of percent identity between any two sequences can be accomplished using a mathematical algorithm. Preferably, naturally or non-naturally occurring variants of IL-2 have amino acid sequences that are at least 70%, preferably 80%, more preferably, 85%, 90%, 91%, 92%, 93%, 94% or 95% identical to the amino acid sequence to the reference molecule, for example, the native human IL-2, or to a shorter portion of the reference IL-2 molecule. More preferably, the molecules are 96%, 97%, 98% or 99% identical. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2:482-489. A variant may, for example, differ by as few as 1 to 10 amino acid residues, such as 6-10, as few as 5, as few as 4, 3, 2, or even 1 amino aid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least twenty (20) contiguous amino acid residues, and may be 30, 40, 50, or more amino acid residues. Corrections for sequence identity associated with conservative residue substitutions or gaps can be made (see Smith-Waterman homology search algorithm).

The precise chemical structure of a polypeptide having IL-2 activity depends on a number of factors. As ionizable amino and carboxyl groups are present in the molecule, a particular polypeptide may be obtained as an acidic or basic salt, or in neutral-form. All such preparations that retain their biological activity when placed in suitable environmental conditions are included in the definition of polypeptides having IL-2 activity as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an IL-2 polypeptide used herein so long as the IL-2 activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of IL-2 polypeptides of interest as used herein.

The art provides substantial guidance regarding the preparation and use of polypeptide variants. In preparing the IL-2 variants, one of skill in the art can readily determine which modifications to the native protein nucleotide or amino acid sequence will result in a variant that is suitable for use as a therapeutically active component of a pharmaceutical composition used in the methods of the present invention.

The IL-2 or variants thereof for use in the methods of the present invention may be from any source, but preferably is recombinant IL-2. By “recombinant IL-2” is intended interleukin-2 that has comparable biological activity to native-sequence IL-2 and that has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for IL-2 is cloned and then expressed in transformed organisms, preferably a microorganism, and most preferably E. coli, as described herein. The host organism expresses the foreign gene to produce IL-2 under expression conditions. Synthetic recombinant 1L-2 can also be made in eukaryotes, such as yeast or human cells. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

For examples of variant IL-2 proteins, see European Patent (EP) Publication No. EP 136,489 (which discloses one or more of the following alterations in the amino acid sequence of naturally occurring IL-2: Asn26 to Gln26; Trp121 to Phe121; Cys58 to Ser58 or Ala58, Cys105 to Ser105 or Ala105; Cys125 to Ser125 or Ala125; deletion of all residues following Arg 120; and the Met-I forms thereof); and the recombinant IL-2 muteins described in European Patent Application No. 83306221.9, filed Oct. 13, 1983 (published May 30, 1984 under Publication No. EP 109,748), which is the equivalent to Belgian Patent No. 893,016, and commonly owned U.S. Pat. No. 4,518,584 (which disclose recombinant human IL-2 mutein wherein the cysteine at position 125, numbered in accordance with native human IL-2, is deleted or replaced by a neutral amino acid; alanyl-ser125-IL-2; and des-alanayl-ser125-IL-2). See also U.S. Pat. No. 4,752,585 (which discloses the following variant IL-2 proteins: ala104 ser125 IL-2, ala104 IL-2, ala104 ala125 IL-2, val104 ser125 IL-2, val104 IL-2, val104 ala125 IL-2, des-ala1 ala104 ser125 IL-2, des-ala1 ala104 IL-2, des-ala1 ala104 ala125 IL-2, des-ala1 val104 ser125 IL-2, des-ala1 val104 IL-2, des-ala1 val104 ala125 IL-2, des-ala1 des-pro2 ala104 ser125 IL-2, des-ala1 des-pro2 ala104 IL-2, des-ala1 des-pro2 ala104 ala125 IL-2, des-ala1 des-pro2 val104 ser125 IL-2, des-ala1 des-pro2 val104 IL-2, des-ala1 des-pro2 val104 ala125 IL-2, des-ala1 des-pro2 des-thr3 ala104 ser125 IL-2, des-ala1 des-pro2 des-thr3 ala104 IL-2, des-ala1 des-pro2 des-thr3 ala104 ala125 IL-2, des-ala1 des-pro2 des-thr3 val104 ser125 IL-2, des-ala1 des-pro2 des-thr3 val104 IL-2, des-ala1 des-pro2 des-thr3 val104 ala125 IL-2, des-ala1 des-pro2 des-thr3 des-ser4 ala104 ser125 IL-2, des-ala1 des-pro2 des-thr3 des-ser4 ala104 IL-2, des-ala1 des-pro2 des-thr3 des-ser4 ala104 ala125 IL-2, des-ala1 des-pro2 des-thr3 des-ser4 val104 ser125 IL-2, des-ala1 des-pro2 des-thr3 des-ser4 val104 IL-2, des-ala1 des-pro2 des-thr3 des-ser4 val104 ala125 IL-2, des-ala1 des-pro2 des-thr3 des-ser4 des-ser5 ala104 ser125 IL-2, des-ala1 des-pro2 des-thr3 des-ser4 des-ser5 ala104 IL-2, des-ala1 des-pro2 des-thr3 des-ser4 des-ser5 ala104 ala125 IL-2, des-ala1 des-pro2 des-thr3 des-ser4 des-ser5 val104 ser125 IL-2, des-ala1 des-pro2 des-thr3 des-ser4 des-ser5 val104 IL-2, des-ala1 des-pro2 des-thr3 des-ser4 des-ser5 val104 ala125 IL-2, des-ala1 des-pro2 des-thr3 des-ser4 des-ser5 des-ser6 ala104 ala125 IL-2, des-ala1 des-pro2 des-thr3 des-ser4 des-ser5 des-ser6 ala104 IL-2, des-ala1 des-pro2 des-thr3 des-ser4 des-ser5 des-ser6 ala104 ser125 IL-2, des-ala1 des-pro2 des-thr3 des-ser4 des-ser5 des-ser6 val104 ser125 IL-2, des-ala1 des-pro2 des-thr3 des-ser4 des-ser5 des-ser6 val104 IL-2, and des-ala1 des-pro2 des-thr3 des-ser4 des-ser5 des-ser6 val104 ala125 IL-2) and U.S. Pat. No. 4,931,543 (which discloses the IL-2 mutein des-alanyl-1, serine-125 human IL-2 used in the examples herein, as well as the other IL-2 muteins).

Also see European Patent Publication No. EP 200,280 (published Dec. 10, 1986), which discloses recombinant IL-2 muteins wherein the methionine at position 104 has been replaced by a conservative amino acid. Examples include the following muteins: ser4 des-ser5 ala104 IL-2; des-ala1 des-pro2 des-thr3 des-ser4 des-ser5 ala104 ala125 IL-2; des-ala1 des-pro2 des-thr3 des-ser4 des-ser5 glu104 ser125 IL-2; des-ala1 des-pro2 des-thr3 des-ser4 des-ser5 glu104 IL-2; des-ala1 des-pro2 des-thr3 des-ser4 des-ser5 glu104 ala125 IL-2; des-ala1 des-pro2 des-thr3 des-ser4 des-ser5 des-ser6 ala104 ala125 IL-2; des-ala1 des-pro2 des-thr3 des-ser4 des-ser5 des-ser6 ala104 IL-2; des-ala1 des-pro2 des-thr3 des-ser4 des-ser5 des-ser6 ala104 ser125 IL-2; des-ala1 des-pro2 des-thr3 des-ser4 des-ser5 des-ser6 glu104 ser125 IL-2; des-ala1 des-pro2 des-thr3 des-ser4 des-ser5 des-ser6 glu104 IL-2; and des-ala1 des-pro2 des-thr3 des-ser4 des-ser5 des-ser6 glu104 ala125 IL-2. See also European Patent Publication No. EP 118,617 and U.S. Pat. No. 5,700,913, which disclose unglycosylated human IL-2 variants bearing alanine instead of native IL-2's methionine as the N-terminal amino acid; an unglycosylated human IL-2 with the initial methionine deleted such that proline is the N-terminal amino acid; and an unglycosylated human IL-2 with an alanine inserted between the N-terminal methionine and proline amino acids.

Other IL-2 muteins include the those disclosed in WO 99/60128 (substitutions of the aspartate at position 20 with histidine or isoleucine, the asparagine at position 88 with arginine, glycine, or isoleucine, or the glutamine at position126 with leucine or gulatamic acid), which reportedly have selective activity for high affinity IL-2 receptors expressed by cells expressing T cell receptors in preference to NK cells and reduced IL-2 toxicity; the muteins disclosed in U.S. Pat. No. 5,229,109 (substitutions of arginine at position 38 with alanine, or substitutions of phenylalanine at position 42 with lysine), which exhibit reduced binding to the high affinity IL-2 receptor when compared to native IL-2 while maintaining the ability to stimulate LAK cells; the muteins disclosed in International Publication No. WO 00/58456 (altering or deleting a naturally occurring (x)D(y) sequence in native IL-2 where D is aspartic acid, (x) is leucine, isoleucine, glycine, or valine, and (y) is valine, leucine or serine), which are claimed to reduce vascular leak syndrome; the IL-2 p1-30 peptide disclosed in International Publication No. WO 00/04048 (corresponding to the first 30 amino acids of IL-2, which contains the entire a-helix A of IL-2 and interacts with the b chain of the IL-2 receptor), which reportedly stimulates NK cells and induction of LAK cells; and a mutant form of the IL-2 p1-30 peptide also disclosed in WO 00/04048 (substitution of aspartic acid at position 20 with lysine), which reportedly is unable to induce vascular bleeds but remains capable of generating LAK cells. Additionally, IL-2 can be modified with polyethylene glycol to provide enhanced solubility and an altered pharmokinetic profile (see U.S. Pat. No. 4,766,106).

The term IL-2 as used herein is also intended to include IL-2 fusions or conjugates comprising IL-2 fused to a second protein or covalently conjugated to polyproline or a water-soluble polymer to reduce dosing frequencies or to improve IL-2 tolerability. For example, the IL-2 (or a variant thereof as defined herein) can be fused to human albumin or an albumin fragment using methods known in the art (see WO 01/79258). Alternatively, the IL-2 can be covalently conjugated to polyproline or polyethylene glycol homopolymers and polyoxyethylated polyols, wherein the homopolymer is unsubstituted or substituted at one end with an alkyl group and the poplyol is unsubstituted, using methods known in the art (see, for example, U.S. Pat. Nos. 4,766,106, 5,206,344, and 4,894,226).

Any pharmaceutical composition comprising IL-2 as the therapeutically active component can be used in the methods of the invention. Such pharmaceutical compositions are known in the art and include, but are not limited to, those disclosed in U.S. Pat. Nos. 4,745,180; 4,766,106; 4,816,440; 4,894,226; 4,931,544; and 5,078,997;

herein incorporated by reference. Thus liquid, lyophilized, or spray-dried compositions comprising IL-2 or variants thereof that are known in the art may be prepared as an aqueous or nonaqueous solution or suspension for subsequent administration to a subject in accordance with the methods of the invention. Each of these compositions will comprise IL-2 or variants thereof as a therapeutically or prophylactically active component. By “therapeutically or prophylactically active component” is intended the IL-2 or variants thereof is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

In preferred embodiments of the invention, the IL-2 containing pharmaceutical compositions useful in the methods of the invention are compositions comprising stabilized monomeric IL-2 or variants thereof, compositions comprising multimeric IL-2 or variants thereof, and compositions comprising stabilized lyophilized or spray-dried IL-2 or variants thereof.

Pharmaceutical compositions comprising stabilized monomeric IL-2 or variants thereof are disclosed in International Publication No. WO 01/24814, entitled “Stabilized Liquid Polypeptide-Containing Pharmaceutical Compositions.” By “monomeric” IL-2 is intended the protein molecules are present substantially in their monomer form, not in an aggregated form, in the pharmaceutical compositions described herein. Hence covalent or hydrophobic oligomers or aggregates of IL-2 are not present. Briefly, the IL-2 in these liquid compositions is formulated with an amount of an amino acid base sufficient to decrease aggregate formation of IL-2 during storage. The amino acid base is an amino acid or a combination of amino acids, where any given amino acid is present either in its free base form or in its salt form. Preferred amino acids are selected from the group consisting of arginine, lysine, aspartic acid, and glutamic acid. These compositions further comprise a buffering agent to maintain pH of the liquid compositions within an acceptable range for stability of IL-2, where the buffering agent is an acid substantially free of its salt form, an acid in its salt form, or a mixture of an acid and its salt form. Preferably the acid is selected from the group consisting of succinic acid, citric acid, phosphoric acid, and glutamic acid. Such compositions are referred to herein as stabilized monomeric IL-2 pharmaceutical compositions.

The amino acid base in these compositions serves to stabilize the IL-2 against aggregate formation during storage of the liquid pharmaceutical composition, while use of an acid substantially free of its salt form, an acid in its salt form, or a mixture of an acid and its salt form as the buffering agent results in a liquid composition having an osmolarity that is nearly isotonic. The liquid pharmaceutical composition may additionally incorporate other stabilizing agents, more particularly methionine, a nonionic surfactant such as polysorbate 80, and EDTA, to further increase stability of the polypeptide. Such liquid pharmaceutical compositions are said to be stabilized, as addition of amino acid base in combination with an acid substantially free of its salt form, an acid in its salt form, or a mixture of an acid and its salt form, results in the compositions having increased storage stability relative to liquid pharmaceutical compositions formulated in the absence of the combination of these two components.

These liquid pharmaceutical compositions comprising stabilized monomeric IL-2 may either be used in an aqueous liquid form, or stored for later use in a frozen state, or in a dried form for later reconstitution into a liquid form or other form suitable for administration to a subject in accordance with the methods of present invention. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Other examples of IL-2 formulations that comprise IL-2 in its nonaggregated monomeric state include those described in Whittington and Faulds (1993) Drugs 46(3):446-514. These formulations include the recombinant IL-2 product in which the recombinant IL-2 mutein Teceleukin (unglycosylated human IL-2 with a methionine residue added at the amino-terminal) is formulated with 0.25% human serum albumin in a lyophilized powder that is reconstituted in isotonic saline, and the recombinant IL-2 mutein Bioleukin (human IL-2 with a methionine residue added at the amino-terminal, and a substitution of the cysteine residue at position 125 of the human IL-2 sequence with alanine) formulated such that 0.1 to 1.0 mg/ml IL-2 mutein is combined with acid, wherein the formulation has a pH of 3.0 to 4.0, advantageously no buffer, and a conductivity of less than 1000 mmhos/cm (advantageously less than 500 mmhos/cm). See EP 373,679; Xhang et al. (1996) Pharmaceut. Res. 13(4):643-644; and Prestrelski et al. (1995) Pharmaceut. Res. 12(9):1250-1258.

Examples of pharmaceutical compositions comprising multimeric IL-2 are disclosed in commonly owned U.S. Pat. No. 4,604,377. By “multimeric” is intended the protein molecules are present in the pharmaceutical composition in a microaggregated form having an average molecular association of 10-50 molecules. These multimers are present as loosely bound, physically associated IL-2 molecules. A lyophilized form of these compositions is available commercially under the tradename Proleukin® IL-2 (Chiron Corporation, Emeryville, Calif.). The lyophilized formulations disclosed in this reference comprise selectively oxidized, microbially produced recombinant IL-2 in which the recombinant IL-2 is admixed with a water soluble carrier such as mannitol that provides bulk, and a sufficient amount of sodium dodecyl sulfate to ensure the solubility of the recombinant IL-2 in water. These compositions are suitable for reconstitution in aqueous injections for parenteral administration and are stable and well tolerated in human patients. When reconstituted, the IL-2 retains its multimeric state. Such lyophilized or liquid compositions comprising multimeric IL-2 are encompassed by the methods of the present invention. Such compositions are referred to herein as multimeric IL-2 pharmaceutical compositions.

The methods of the present invention may also use stabilized lyophilized or spray-dried pharmaceutical compositions comprising IL-2, which may be reconstituted into a liquid or other suitable form for administration in accordance with methods of the invention. Such pharmaceutical compositions are disclosed in International Publication No. WO 01/49274 entitled “Methods for Pulmonary Delivery of Interleukin-2.” These compositions may further comprise at least one bulking agent, at least one agent in an amount sufficient to stabilize the protein during the drying process, or both. By “stabilized” is intended the IL-2 protein or variants thereof retains its monomeric or multimeric form as well as its other key properties of quality, purity, and potency following lyophilization or spray-drying to obtain the solid or dry powder form of the composition. In these compositions, preferred carrier materials for use as a bulking agent include glycine, mannitol, alanine, valine, or any combination thereof, most preferably glycine. The bulking agent is present in the formulation in the range of 0% to about 10% (w/v), depending upon the agent used. Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%. These stabilized lyophilized or spray-dried compositions may optionally comprise methionine, ethylenediaminetetracetic acid (EDTA) or one of its salts such as disodium EDTA or other chelating agent, which protect the IL-2 or variants thereof against methionine oxidation. Use of these agents in this manner is described in U.S. application Ser. No. 09/677,643, herein incorporated by reference. The stabilized lyophilized or spray-dried compositions may be formulated using a buffering agent, which maintains the pH of the pharmaceutical composition within an acceptable range, preferably between about pH 4.0 to about pH 8.5, when in a liquid phase, such as during the formulation process or following reconstitution of the dried form of the composition. Buffers are chosen such that they are compatible with the drying process and do not affect the quality, purity, potency, and stability of the protein during processing and upon storage.

The previously described stabilized monomeric, multimeric, and stabilized lyophilized or spray-dried IL-2 pharmaceutical compositions represent suitable compositions for use in the methods of the invention. However, any pharmaceutical composition comprising IL-2 as a therapeutically active component is encompassed by the methods of the invention.

As used herein, the term “anti-HER2 antibody” encompasses any antibody that specifically recognizes and specifically binds to the HER2 protein, including polyclonal anti-HER2 antibodies, monoclonal anti-HER2 antibodies, human anti-HER2 antibodies, humanized anti-HER2 antibodies, chimeric anti-HER2 antibodies, xenogenoic anti-HER2 antibodies, and fragments of these anti-HER2 antibodies that specifically recognize and bind to the HER2 protein, preferably to the extracellular domain of the HER2 protein. Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example.

Anti-HER2 antibodies of murine origin and their humanized and chimeric versions are suitable for use in the methods of the present invention. Examples of such anti-HER2 antibodies include, but are not limited to, the 4D5 antibody (described in U.S. Pat. Nos. 5,677,171 and 5,772,997); and the 520C9 antibody and its functional equivalents, designated 452F2, 736G9, 741F8, 758G5, and 761B10 (described in U.S. Pat. No. 6,054,561); herein incorporated by reference.

The term “anti-HER2 antibody” as used herein encompasses chimeric anti-HER2 antibodies. By “chimeric antibodies” is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the HER2 protein. The non-human source can be any vertebrate source that can be used to generate antibodies to a human cell surface antigen of interest or material comprising a human cell surface antigen of interest. Such non-human sources include, but are not limited to, rodents (e.g., rabbit, rat, mouse, etc.; see, for example, U.S. Pat. No. 4,816,567, herein incorporated by reference) and non-human primates (e.g., Old World Monkey, Ape, etc.; see, for example, U.S. Pat. Nos. 5,750,105 and 5,756,096; herein incorporated by reference). Most preferably, the non-human component (variable region) is derived from a murine source. Such chimeric antibodies are described in U.S. Pat. Nos. 5,750,105; 5,500,362; 5,677,180; 5,721,108; and 5,843,685; herein incorporated by reference.

Humanized anti-HER2 antibodies are also encompassed by the term anti-HER2 antibody as used herein. By “humanized” is intended forms of anti-HER2 antibodies that contain minimal sequence derived from non-human immunoglobulin sequences. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205; herein incorporated by reference. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al. (1988) Nature 332:323-329; and Presta (1992) Curr. Op. Struct. Biol. 2:593-596; herein incorporated by reference. One such humanized anti-HER2 antibody is commercially available under the tradename Herceptin® (Genentech, Inc., San Francisco, Calif.).

Also encompassed by the term anti-HER2 antibodies are xenogeneic or modified anti-HER2 antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598, herein incorporated by reference.

Fragments of the anti-HER2 antibodies are suitable for use in the methods of the invention so long as they retain the desired affinity of the full-length antibody. Thus, suitable fragments of an anti-HER2 antibody will retain the ability to bind to the HER2 receptor protein, and are herein referred to as “antigen-binding fragments.” Fragments of an antibody comprise a portion of a full-length antibody, generally the antigen binding or variable region thereof. Examples of antibody fragments include, but are not limited to, Fab, Fab′, F(ab′)₂, and Fv fragments and single-chain antibody molecules. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; 5,856,456; herein incorporated by reference. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody phage libraries generated using the techniques described in McCafferty et al. (1990) Nature 348:552-554 (1990). Clackson et al. (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

A humanized antibody has one or more amino acid residues introduced into it from a source that is non-human. These non-human amino acid residues are often referred to as “donor” residues, which are typically taken from a “donor” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent CDRs or CDR sequences for the corresponding sequences of a human antibody. See, for example, U.S. Pat. Nos. 5,225,539; 5,585,089; 5,693,761; 5,693,762; 5,859,205; herein incorporated by reference. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; and 5,859,205. See also U.S. Pat. No. 6,180,370, and International Publication No. WO 01/27160, where humanized antibodies and techniques for producing humanized antibodies having improved affinity for a predetermined antigen are disclosed.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

Alternatively, methods for producing proteins that have reduced immunogenic response may be used to generate anti-HER2 antibodies suitable for use in the methods of the present invention. See, for example, the methods disclosed in WO 98/52976, herein incorporated by reference. Anti-HER2 antibodies generated using such a method are encompassed by the term “anti-HER2 antibody” as used herein.

Further, any of the previously described anti-HER2 antibodies may be conjugated prior to use in the methods of the present invention. Such conjugated antibodies are available in the art. Thus, the anti-HER2 antibody may be labeled using an indirect labeling or indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivagtava and Mease (1991) Nucl. Med. Bio. 18: 589-603, herein incorporated by reference. Alternatively, the anti-HER2 antibody may be labeled using “direct labeling” or a “direct labeling approach”, where a radionuclide is covalently attached directly to an antibody (typically via an amino acid residue). Preferred radionuclides are provided in Srivagtava and Mease (1991) supra. The indirect labeling approach is particularly preferred.

The anti-HER2 antibodies are typically provided by standard technique within a pharmaceutically acceptable buffer, for example, sterile saline, sterile buffered water, propylene glycol, combinations of the foregoing, etc. Methods for preparing parenterally administrable agents are described in Remington 's Pharmaceutical Sciences (18^{th} ed.; Mack Pub. Co.: Eaton, Pa., 1990), herein incorporated by reference. See also, for example, WO 98/56418, which describes stabilized antibody pharmaceutical formulations suitable for use in the methods of the present invention.

The following examples are offered by way of illustration and not by way of limitation.

### EXPERIMENTAL

### Example 1

### Phase I Dose Escalation Study with Weekly Trastuzamab Therapy in Combination with Constant IL-2 Dosing Regimen of L2-7001 IL-2 in Subjects with Her-Her-2/Neu-Positive Metastatic Breast Cancer

A phase I study is carried out to examine the use of a monomeric formulation of IL-2, L2-7001, in combination with weekly administration of trastuzamab (Herceptin®) for the treatment of subjects with he r-2/neu-positive metastatic breast cancer. L2-7001 is a liquid formulation that comprises the same human IL-2 mutein (aldesleukin) as Proleukin® IL-2 with the exception of the final purification steps prior to its formulation. As noted below in Example 2, this IL-2 mutein is expressed from E. coli. The initial purification steps to obtain aldesleukin are similar for the two formulations. See U.S. Pat. No. 4,931,543. In both cases, the recombinantly produced IL-2 mutein occurs as refractile bodies within the host cells. Following cell disruption, the refractile bodies are isolated and initially purified using size exclusion chromatography and RP-HPLC. The remaining purification steps for the IL-2 mutein used in L2-7001 are as follows. The resulting protein precipitate is solubilized by guanidine hydrochloride, then processed by diafiltration, ion exchange chromatography, and subsequent diafiltration to obtain the final purified IL-2 mutein for use in making the L2-7001 formulation. In contrast, when this IL-2 mutein is used in Proleukin® IL-2, the protein precipitate resulting from the initial purification steps is solubilized by 1% SDS, then processed by size exclusion chromatography and dialfiltration. The purified IL-2 mutein is then formulated into L2-7001 according to the method disclosed in the copending application entitled “Stabilized Liquid Polypeptide-Containing Pharmaceutical Compositions,” filed Oct. 3, 2000, and assigned U.S. application Ser. No. 09/677,643.

The anti-HER antibody administered in this study is commercially available as Herceptin® (trastuzumab; Genentech, Inc., San Francisco, Calif.). Herceptin® is a recombinant humanized monoclonal antibody that selectively binds to the extracellular domain of the human epidermal growth factor receptor 2 protein, HER2. The antibody is an IgG₁ kappa that contains human framework regions with the complementarity-determining regions of a murine antibody (4D5) that binds to HER2. The humanized antibody against HER2 is produced by a mammalian cell (Chinese hamster ovary (CHO)) suspension culture in a nutrient medium containing the antibiotic gentamicin. This antibiotic is not detectable in the final product.

Herceptin® is a sterile, white to pale yellow, preservative-free lyophilized powder for intravenous (IV) administration. The nominal content of each Herceptin® vial is 440 mg trastuzumab, 9.9 mg L-histidine HCl, 6.4 mg L-histidine, 400 mg aa-trehalose dihydrate, and 1.8 mg polysorbate 20, USP.

Study Description

This is an MTD dose-finding study in which a constant total weekly dose of L2-7001 is administered over a 4-week period in combination with weekly doses of Trastuzamab. Trastuzamab is given at the labeled dose of 4 mg/kg at week 1, followed by a weekly infusion of 2 mg/kg for 5 weeks. The total weekly doses of L2-7001 are partitioned into three equivalent doses that are administered subcutaneously according to a three-times-a-week dosing schedule, with a minimum of 48 hours between administrations. L2-7001 administration begins at week 2 and continues for 4 weeks (i.e., through week 5 of the study). The initial L2-7001 total weekly doses to be studied are 270 μg, 540 μg, and 810 μg, and 1080 μg.

Weeks 2 through 6 represent the primary MTD evaluation period. Cohorts of three to six subjects are enrolled at each L2-7001 dose level, and each subject participates in only one cohort. Each dose group is treated and observed through the end of week 6 before treatment of subjects at the next higher dose level of L2-7001 can begin. An individual who experiences a DLT at any dose level has both drugs (L2-7001 and Trastuzumab) stopped and is monitored closely thereafter. L2-7001 may be stopped for a maximum of two weeks. If more than two weeks have elapsed since L2-7001 was stopped, the subject will be terminated from the study. When all signs of toxicity (other than congestive heart failure or left ventricular dysfunction) are reduced to at least a grade 2, the subject may be restarted at the next lower dose level of L2-7001 (or discontinued if the subject was treated at a total weekly dose of 270 μg). The MTD is defined as the highest dose at which a minimum of six evaluable subjects have been treated with the 6-week regimen and DLTs have occurred in no more than one subject during weeks 2 to 6. If a DLT is observed in only one subject in a cohort of less than six subjects, additional subjects may be enrolled up to a total of six subjects at this dose level and dose escalation will only proceed if no more than one subject has experienced DLT during weeks 2 to 6. If DLTs are observed in two or more subjects at any dose level, that dose will be considered to be above the MTD, and additional subjects will be enrolled at the previous lower dose if that dose level previously had less than six subjects enrolled. If the MTD has not been reached at the 1080 μg total weekly dose, additional dose levels may be added in increments of 90 μg until MTD is established (e.g., next total weekly dose would be 1350 μg, and then 1620 μg).

After week 6, subjects have the option of continuing additional 5-week cycles of L2-7001 and trastuzumab at their current dose level until disease progression. Each additional cycle of L2-7001/trastuzumab consists 4 weeks of L2-7001 at the assigned dose followed by a 1-week holiday from IL-2 dosing (i.e., IL-2 dosing is withheld for 1 week), with trastuzumab given weekly throughout each cycle. Thus, if a first optional cycle is initiated at the conclusion of week 6 of the study, trastuzumab is administered on day 1 of weeks 7, 8, 9, 10, and 11, and the assigned total weekly dose of L2-7001 IL-2 is partitioned into three equivalent doses to be administered during weeks 7, 8, 9, and 10 according to a three-times-a-week dosing schedule.

Once the MTD is determined, any subject who did not experience a DLT by week 6 at the L2-7001 dose level above the MTD will continue their L2-7001 at the MTD. The cohort receiving the MTD will be expanded to a total of 30 subjects to gain more safety data and to evaluate NK cell expansion and tumor response at the MTD.

Safety and efficacy are evaluated during weekly outpatient visits. Tumor response is based on assessment of all tumor sites obtained by CT scan, other imaging tools, or by physical examination. Cardiac function is closely monitored by physical examination and ECHO/MUGA throughout the study. DLTs will be defined as cardiac adverse events or any other NCI grade 3 or 4 treatment-related adverse reactions including pulmonary reactions, with the exception of hematologic laboratory abnormalities and fever, which require a grade 4 toxicity to be considered a DLT.

Selection of Study Population

The primary inclusion criteria are: documentation of her-2/neu positive breast cancer (IHC 3+ or FISH+) with measurable disease per the clinical investigators. Subjects will be excluded from the study for the following reasons: evidence of central nervous system (CNS) metastases or carcinomatous meningitis at the start of the study; previous or concurrent additional malignancy except inactive non-melanoma skin cancer, in situ carcinoma of the cervix, or other solid tumor treated curatively, and without evidence of recurrence for at least two years immediately prior to study entry; symptomatic thyroid disease requiring medical intervention other than replacement treatment for hypothyroidism; history of autoimmune disease; therapy with prohibited medications prior to study entry; serious uncontrolled active infections; type I hypersensitivity or anaphylactic reactions to murine proteins; history of cardiac dysfunction or an abnormal echo or MUGA scan; intolerance to trastuzamab.

Safety and Efficacy

Safety and efficacy measurements are carried out during weekly outpatient visits. Safety is evaluated from the incidence of all adverse events (including serious adverse events [SAEs]). The severity of adverse events will be graded according the NCI common toxicity grading scale. The incidence of hematology, chemistry, and urinalysis values outside the normal range is determined at baseline and at weeks 1, 2, 3, 4, 6, 8, 9 and 17 evaluations. Echo or MUGA scan is done at the baseline and week 17.

Efficacy is measured by tumor response at weeks 5, 9 and 17. Responses are classified by the investigator according to the Response Evaluation Criteria in Solid Tumors (RECIST) guidelines set forth by the EORTC, NCI and the National Cancer Institute of Canada Clinical Trials Group, as complete response (CR), partial response (PR), stable disease (SD), or progressive disease (PD). Responses at week 9 or week 17 are confirmed with repeat evaluation 4 weeks later. Responders and subjects with stable disease at weeks 5 and 9 continue receiving trastuzamab weekly and are followed every 8 weeks for tumor signs and symptoms and with physical examination and CT scans until disease progression (i.e. progressive disease).

Criteria for Response, Progression and Relapse

Tumor response is evaluated according to Response Evaluation Criteria in Solid Tumors (RECIST) (see Therasse et al. (2000) J. Natl. Cancer Inst. 92: 205-216). Grading of tumor response is as follows:

Complete response—Documentation of the complete disappearance of all target lesions and non-target lesions (or of all symptoms and signs of disease) and normalization of tumor marker level.

Partial response—At least a 30% decrease in the sum of the longest diameter (LD) of target lesions, taking as reference the baseline sum LD, no significant increase in non-target lesions and no new lesions

Stable disease—Neither sufficient shrinkage to qualify for PR nor sufficient increase to qualify for PD, taking as reference the smallest sum LD since the treatment started.

Progressive disease—At least 20% increase in the sum of the LD of target lesions, taking as reference the smallest sum LD recorded since the treatment started or the appearance of one or more new lesions or unequivocal progression of existing non-target lesions.

### Example 2

### Weekly Trastuzamab Therapy in Combination with 8-Week Two-Level IL-2 Dosing Regimen of Proleukin® in Subjects with Her-2/Neu-Positive Metastatic Breast Cancer

The objective of this study is to evaluate the tumor response after thrice-weekly administration of Proleukin® IL-2 (aldesleukin) in combination with weekly administration of trastuzamab (Herceptin®) when administered to subjects with her-2/neu positive breast cancer, the study will define the safety and tolerability of thrice-weekly administration of Proleukin® IL-2 in combination with weekly administration of trastuzamab.

The IL-2 formulation used in this study is manufactured by Chiron Corporation of Emeryville, Calif., under the tradename Proleukin® IL-2. The IL-2 in this formulation is a recombinantly produced, unglycosylated human IL-2 mutein, called aldesleukin, which differs from the native human IL-2 amino acid sequence in having the initial alanine residue eliminated and the cysteine residue at position 125 replaced by a serine residue (referred to as des-alanyl-1, serine-125 human interleukin-2). This IL-2 mutein is expressed from E. coli, and subsequently purified by diafiltration and cation exchange chromatography as described in U.S. Pat. No. 4,931,543. The IL-2 formulation marketed as Proleukin® IL-2 is supplied as a sterile, white to off-white preservative-free lyophilized powder in vials containing 1.3 mg of protein (22 MIU).

Study Description

Eligible subjects are administered Proleukin® IL-2 by subcutaneous injection and administered trastuzamab by IV infusion. The dose of trastuzamab used is according to its label. An initial loading dose of 4 mg/kg of trastuzamab is used on day 1 followed by 2 mg/kg weekly until disease progression or antibody toxicity. The total weekly dose of Proleukin® IL-2 is 42.0 MIU during week 2 through week 5. This 42.0 MIU dose is partitioned into three equivalent doses that are administered according to a three-times-a-week dosing schedule (i.e., each equivalent dose is 14.0 MIU). The total weekly dose of Proleukin® IL-2 is 30.0 MIU during week 6 to week 9. This 30.0 MIU dose is partitioned into three equivalent doses that are administered according to a three-times-a-week dosing schedule (i.e., each equivalent dose is 10.0 MIU). Patients are monitored for efficacy and safety of this treatment regimen throughout the 9-week treatment period, with follow-up determinations occurring at week 17 post-initiation of the study.

### Example 3

### Weekly Trastuzumab Therapy in Combination with 8-week Two-Dose Regimen of L2-7001 in Subjects with Her2/neu Positive Metastatic Breast Cancer

As an alternative to the dosing regimen outlined in Example 2, eligible subjects are administered the monomeric IL-2 formulation L2-7001 IL-2, instead of Proleukin® IL-2. Trastuzamab is given at the labeled dose of 4 mg/kg at week 1, followed by a weekly infusion of 2 mg/kg for 8 weeks. Subjects begin concomitant administration of L2-7001 IL-2 by subcutaneous injection on day 1 of the second week (i.e., day 8 of the treatment period). The total weekly dose of L2-7001 IL-2 is partitioned into three equivalent doses that are administered according to a three-times-a-week dosing schedule, with a minimum of 48 hours between administrations, for a period of 8 weeks (i.e., total of 24 doses during weeks 2-9 of the treatment period). During weeks 2-5, the total weekly dose of L2-7001 IL-2 to be administered as three equivalent doses is 810 μg (i.e., each equivalent dose is 270 μg). After 4 weeks of L2-7001 IL-2 administration, the total weekly dose of L2-7001 IL-2 is lowered to 540 μg. Thus, during weeks 6-9, a total weekly dose of 540 μg L2-7001 IL-2 is partitioned into three equivalent doses (i.e., each 180 μg) that are administered according to the three-times-per-week dosing schedule. Subjects are monitored for efficacy and safety of this treatment regimen throughout the 9-week treatment period, with follow-up determinations occurring through week 16 (i.e., for 7 weeks beyond the last week of IL-2 administration).

### Example 4

### Calculation IL-2 Serum Concentration-Time Curves for Pharmaceutical Formulations of IL-2

The area under the serum concentration-time curve (AUC) of Proleukin® IL-2 administered subcutaneously (SC) at 4.5 million international units (MIU) (equivalent to approximately 275 μg protein) was determined using data from an unpublished HIV study. Serum concentration time profiles were measured in 8 IL-2 naive, HIV patients following an initial exposure to IL-2 dosing in this study. For each patient, the AUC was calculated using the linear trapezoidal rule up to the last measurable concentrations and extrapolated to 24 hours (Winnonlin software version 3.1, Pharsight Corporation, Calif.). The average AUC₀₋₂₄, SD, and the lower and upper 95% confidence limits at 4.5 MIU dose are presented in Table 2.

The AUC₀₋₂₄ value of Proleukin® IL-2 administered SC at doses equivalent to 18 MIU (1100 μg) was estimated using data from three different studies where this IL-2 product was administered SC. Two are published studies, one in HIV patients (N 3) (Piscitelli et al. (1996) Pharmacotherapy 16(5):754-759) and one in cancer patients (N=7) (Kirchner et al. (1998) Br. J Clin. Pharmacol. 46:5-10). The third is an unpublished study in which serum concentration time data were available from 6 cancer patients after SC doses of IL-2. The similarity of the AUC in cancer and HIV patients was previously established (unpublished data). The actual doses administered in these three studies ranged between 18 and 34 MIU. For the two published trials, the AUC up to 24 hours (AUC₀₋₂₄) values were normalized to 18 MIU dose by multiplying the AUC with the quotient of 18 and actual dose in MIU. For example, if the AUC₀₋₂₄ for a 20 MIU dose was calculated to be 400, the normalized AUC₀₋₂₄ would be 400·18/20=360. For the unpublished cancer-patient study, individual AUC values were calculated from the serum concentration time data using the linear trapezoidal rule up to the last measurable concentrations and extrapolated to. 24 hours (Winnonlin software version 3.1, Pharsight Corporation, California) then were normalized to 18 MW dose as noted above. The overall mean and SD for all three studies was calculated as the weighted average of the means and variances, respectively, using equations 1 and 2.

$$\begin{matrix}1. {\overline{X}}_{p} = \frac{( {n}_{1} {\overline{X}}_{1} + {n}_{2} {\overline{X}}_{2} + {n}_{3} {\overline{X}}_{3} )}{( {n}_{1} + {n}_{2} + {n}_{3} )} \\ 2. {SD}_{p} = \sqrt{\frac{( {n}_{1} - 1 ) {s}_{1}^{2} + ( {n}_{2} - 1 ) {s}_{2}^{2} + ( {n}_{3} - 1 ) {s}_{3}^{2}}{( {n}_{1} + {n}_{2} + {n}_{3} - 3 )}}\end{matrix}$$

Where n₁,n₂,n₃,\overline{X}₁, \overline{X}₂, \overline{X}₃ and s₁²,s₂²,s₃² are the number of subjects, means, and variances for each of the three studies, respectively. \overline{X}_{P} and SD_{P} are estimates of the overall mean and standard deviation. The overall average AUC, SD, and the lower and upper 95% confidence limits at 18 MIU are also presented in Table 2.

TABLE 2
Average (±SD) AUC₀₋₂₄ obtained after initial exposure to a single dose
administration of Proleukin ® IL-2 administered subcutaneously.

| Proleukin ® IL-2 | | | | |
|---|---|---|---|---|
| Dose | AUC₀₋₂₄ | | LL of | UL of |
| (MIU/μg) | (IU*hr/ml) | SD | 95% CI¹ | 95% CI¹ |
| 4.5/275 | 51 | 14 | 23 | 78 |
| 6.0/367² | 65 | | 22 | 107 |
| 7.5/458 | 79 | 29 | 21 | 137 |
| 18/1100 | 344 | 127 | 90 | 598 |

¹Upper (UL) and lower (LL) limits of the 95% confidence intervals (CI). 95% CI were calculated as the mean ± 2 SD.
²Values for 6.0 MIU are estimated based on actual values for 4.5 MIU and 7.5 MIU.

Similar to Proleukin® IL-2, L2-7001, a liquid formulation of monomeric IL-2, was administered to HIV patients at doses ranging from 50 to 180 μg (unpublished data). The exposures obtained from this study as measured by AUC are shown in Table 3. These exposure values were within the range of the exposure values generated using Proleukin® IL-2 (Table 2).

TABLE 3
Average (±SD) AUC₀₋₂₄ obtained after an initial exposure to a single dose
administration of the monomeric IL-2 formulation L2-7001.

| L2-7001 Dose | AUC₀₋₂₄ | |
|---|---|---|
| MIU/μg | (IU*hr/ml) | SD |
| 0.82/50 | 60 | 11 |
| 1.5/90 | 110 | 36 |
| 2.2/135 | 143 | 41 |
| 2.9/180 | 275 | 99 |

The IL-2 exposure data (AUC) was obtained from the published literature where recombinant human native IL-2 was administered SC to 8 cancer patients at doses ranging from 0.1 MU to 3.0 MU. The reported average (%CV) AUCs for the 0.3, 1, and 3 MU dose levels were 120 (38), 177 (36), and 359 (46) U*hr/ml (Gustavson (1998) J. Biol. Response Modifiers 1998:440-449). As indicated in Thompson et al. 1987 Cancer Research 47:4202-4207, the units measured in this study were normalized to BRMP units (Rossio et al. (1986) Lymphokine Research 5 (suppl 1):S13-S18), which was adopted later as international units (IU) by WHO (Gearing and Thorpe (1988) J. Immunological Methods 114:3-9). The AUC values generated under the study conditions also agree well with the established Proleukin® IL-2 exposure.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the present invention and the embodiments disclosed herein.

That which is claimed:

1. A method of treating a cancer characterized by overexpression of the HER2 receptor protein in a human subject, said method comprising administering to said subject a therapeutically effective dose of an anti-HER2 antibody according to a weekly dosing schedule, or once every three weeks, throughout a treatment period in combination with the administration of one or more cycles of a constant IL-2 dosing regimen during said treatment period, wherein said constant IL-2 dosing regimen comprises a first time period, wherein a constant total weekly dose of IL-2 is administered to said subject, and a second time period, wherein IL-2 administration is withheld from said subject.

2. The method of claim 1, wherein said first time period has a duration of about 2 weeks to about 12 weeks, and wherein said second time period has a duration of about 1 week to about 4 weeks.

3. The method of claim 2, wherein said first time period has a duration of 4 weeks, and wherein said second time period has a duration of 1 week.

4. The method of claim 2, wherein a first administration of said anti-HER2 antibody begins on day 1 of said treatment period, and wherein a first cycle of said constant IL-2 dosing regimen is initiated within 10 days of said first administration of said anti-HER2 antibody.

5. The method of claim 4, wherein said first cycle of said constant IL-2 dosing regimen is initiated on day 8 of said treatment period.

6. The method of claim 4, wherein said treatment period comprises one or more subsequent cycles of said constant IL-2 dosing regimen that is initiated within 4 weeks following completion of said first cycle of said constant TL-2 dosing regimen or completion of any subsequent cycle of said constant IL-2 dosing regimen, wherein said anti-HER2 antibody is administered throughout said treatment period.

7. The method of claim 1, wherein said therapeutically effective dose of said anti-HER2 antibody is in the range from about 1.0 mg/kg to about 10.0 mg/kg.

8. The method of claim 7, wherein said therapeutically effective dose of said anti-HER2 antibody is about 4.0 mg/kg on day 1 of said treatment period and about 2.0 mg/kg for each dose thereafter.

9. The method of claim 1, wherein said constant total weekly dose of IL-2 is administered as a single dose or is partitioned into a series of equivalent doses that are administered according to a two-, three-, four-, five-, six- or seven-times-a-week dosing schedule.

10. The method of claim 9, wherein said IL-2 is administered by a route selected from the group consisting of intravenous, intramuscular, and subcutaneous.

11. The method of claim 1, wherein said constant total weekly dose of IL-2 is in an amount equivalent to a total weekly dose of a reference IL-2 standard in a range from 270 μg to 1620 μg as determined by the area under the serum concentration-time curve from human pharmacokinetic (PK) data.

12. The method of claim 11, wherein said constant total weekly dose of IL-2 is 600 μg to 1080 μg.

13. The method of claim 12, wherein said constant total weekly dose of IL-2 is partitioned into two equivalent doses that are administered according to a two-times-a-week dosing schedule.

14. The method of claim 11, wherein said constant total weekly dose of IL-2 is 360 μg to 840 μg.

15. The method of claim 14, wherein said constant total weekly dose of IL-2 is partitioned into three equivalent doses that are administered according to a three-times-a-week dosing schedule.

16. The method of claim 1, wherein said IL-2 is provided in a pharmaceutical composition selected from the group consisting of a monomeric IL-2 pharmaceutical composition, a multimeric IL-2 pharmaceutical composition, a stabilized lyophilized IL-2 pharmaceutical composition, and a stabilized spray-dried IL-2 pharmaceutical composition.

17. The method of claim 16, wherein said IL-2 is provided in a multimeric IL-2 pharmaceutical composition.

18. The method of claim 17, wherein said constant total weekly dose of IL-2 is an amount of said multimeric IL-2 pharmaceutical composition equivalent to a total weekly dose of IL-2 of about 1100 μg to about 3300 μg.

19. The method of claim 18, wherein said constant total weekly dose of IL-2 is an amount of said multimeric IL-2 pharmaceutical composition equivalent to a total weekly dose of IL-2 of 1833 μg to 3300 μg, and wherein said constant total weekly dose of IL-2 is partitioned into two equivalent doses that are administered according to a two-times-a-week dosing schedule.

20. The method of claim 18, wherein said constant total weekly dose of IL-2 is an amount of said multimeric IL-2 pharmaceutical composition equivalent to a total weekly dose of IL-2 of 1100 μg to 2567 μg, and wherein said constant total weekly dose of IL-2 is partitioned into three equivalent doses that are administered according to a three-times-a-week dosing schedule.

21. The method of claim 18, wherein said therapeutically effective dose of said anti-HER2 antibody is about 4.0 mg/kg on day 1 of said treatment period and about 2.0 mg/kg for each dose thereafter.

22. The method of claim 1, wherein said IL-2 is recombinantly produced IL-2 having an amino acid sequence for human IL-2 or a variant thereof having at least 70% sequence identity to the amino acid sequence for human IL-2.

23. The method of claim 22, wherein said variant there of is des-alanyl-1, serine 125 human interleukin-2.

24. The method of claim 1, wherein said anti-HER2 antibody comprises at least one human constant region.

25. The method of claim 1, wherein said anti-HER2 antibody is selected from the group consisting of 4D5, 520C9, an antigen-binding fragment of 4D5, and an antigen-binding fragment of 520C9.

26. The method of claim 25, wherein said anti-HER2 antibody is 4D5 or a humanized, chimeric, or human form thereof.

27. A method of treating a cancer characterized by overexpression of the HER2 receptor protein in a human subject, said method comprising administering to said subject a therapeutically effective dose of an anti-HER2 antibody according to a weekly dosing schedule, or once every three weeks, throughout a treatment period in combination with the administration of one or more cycles of a two-level IL-2 dosing regimen during said treatment period, wherein said two-level IL-2 dosing regimen comprises a first time period, wherein a higher total weekly dose of IL-2 is administered to said subject, followed by a second time period, wherein a lower total weekly dose of IL-2 is administered to said subject.

28. The method of claim 27, wherein a first dose of IL-2 is administered to aid subject prior to administering a first dose of anti-HER2 antibody.

29. The method of claim 28, wherein said first dose of IL-2 is administered up to one month before the first dose of anti-HER2 antibody is administered to said subject.

30. The method of claim 29, wherein said first dose of IL-2 is administered one week before the first dose of anti-HER2 antibody is administered to said subject.

31. The method of claim 27, wherein a first dose of IL-2 is administered to said subject concurrently with a first dose of anti-HER2 antibody.

32. The method of claim 27, wherein a first dose of IL-2 is administered to said subject one week after a first dose of anti-HER2 antibody is administered to said subject.

33. The method of claim 27, wherein said therapeutically effective dose of said anti-HER2 antibody is in the range from about 1.0 mg/kg to about 10.0 mg/kg.

34. The method of claim 33, wherein said therapeutically effective dose of said anti-HER2 antibody is about 4.0 mg/kg on day 1 of said treatment period and about 2.0 mg/kg for each dose thereafter.

35. The method of claim 27, wherein said two-level dosing regimen of IL-2 has a combined duration of 4 weeks to 16 weeks.

36. The method of claim 35, wherein said first time period of said two-level dosing regimen of IL-2 has a duration of at least 1 week out of said combined duration of 4 weeks to 16 weeks.

37. The method of claim 35, wherein said first time period of said two-level dosing regimen of IL-2 has a duration that is one-half of said combined duration of 4 weeks to 16 weeks.

38. The method of claim 27, wherein said higher total weekly dose of IL-2 is administered as a single dose or is partitioned into a first series of equivalent doses that are administered according to a two-, three-, four-, five-, six- or seven-times-a-week dosing schedule, and wherein said lower total weekly dose of IL-2 is administered as a single dose or is partitioned into a second series of equivalent doses that are administered according to a two-, three-, four-, five-, six- or seven-times-a-week dosing schedule.

39. The method of claim 38, wherein said IL-2 is administered by a route selected from the group consisting of intravenous, intramuscular, and subcutaneous.

40. The method of claim 38, wherein said higher total weekly dose of IL-2 is administered as a single dose.

41. The method of claim 38, wherein said first series of equivalent doses is administered according to a two-times-a-week dosing schedule.

42. The method of claim 38, wherein said first series of equivalent doses is administered according to a three-times-a-week dosing schedule.

43. The method of claim 38, wherein said first series of equivalent doses is administered according to a four-times-a-week dosing schedule.

44. The method of claim 38, wherein said first series of equivalent doses is administered according to a five-times-a-week dosing schedule.

45. The method of claim 38, wherein said first series of equivalent doses is administered according to a six-times-a-week dosing schedule.

46. The method of claim 38, wherein said first series of equivalent doses is administered according to a seven-times-a-week dosing schedule.

47. The method of claim 38, wherein said lower total weekly dose of IL-2 is administered as a single dose.

48. The method of claim 38, wherein said second series of equivalent doses is administered according to a two-times-a-week dosing schedule.

49. The method of claim 38, wherein said second series of equivalent doses is administered according to a three-times-a-week dosing schedule.

50. The method of claim 38, wherein said second series of equivalent doses is administered according to a four-times-a-week dosing schedule.

51. The method of claim 38, wherein said second series of equivalent doses is administered according to a five-times-a-week dosing schedule.

52. The method of claim 38, wherein said second series of equivalent doses is administered according to a six-times-a-week dosing schedule.

53. The method of claim 38, wherein said second series of equivalent doses is administered according to a seven-times-a-week dosing schedule.

54. The method of claim 27, wherein said higher total weekly dose of IL-2 is in an amount equivalent to a total weekly dose of a reference IL-2 standard in a range from 600 μg to 1620 μg as determined by the area under the serum concentration-time curve from human pharmacokinetic (PK) data, and wherein said lower total weekly dose of IL-2 is in an amount equivalent to a total weekly dose of a reference IL-2 standard in a range from 360 μg to about 1080 μg as determined by the area under the serum concentration-time curve from human PK data, and wherein said lower total weekly dose of IL-2 is lower than said higher total weekly dose of IL-2.

55. The method of claim 54, wherein said higher total weekly dose of IL-2 is administered as a single dose or is partitioned into a first series of equivalent doses that are administered according to a two-, three-, four-, five-, six- or seven-times-a-week dosing schedule, and wherein said lower total weekly dose of IL-2 is administered as a single dose or is partitioned into a second series of equivalent doses that are administered according to a two-, three-, four-, five-, six- or seven-times-a-week dosing schedule.

56. The method of claim 54, wherein said higher total weekly dose of IL-2 is 600 μg to 1080 μg and said lower total weekly dose of IL-2 is 360 μg to 840 μg.

57. The method of claim 56, wherein said higher total weekly dose of IL-2 is 1080 μg and said lower total weekly dose of IL-2 is 840 μg.

58. The method of claim 54, wherein said therapeutically effective dose of said anti-HER2 antibody is in the range from about 1.0 mg/kg to about 10.0 mg/kg.

59. The method of claim 58, wherein said therapeutically effective dose of said anti-HER2 antibody is about 4.0 mg/kg on day 1 of said treatment period and about 2.0 mg/kg for each dose thereafter.

60. The method of claim 27, wherein said IL-2 is provided in a pharmaceutical composition selected from the group consisting of a monomeric IL-2 pharmaceutical composition, a multimeric IL-2 pharmaceutical composition, a stabilized lyophilized IL-2 pharmaceutical composition, and a stabilized spray-dried IL-2 pharmaceutical composition.

61. The method of claim 60, wherein said IL-2 is provided in a multimeric IL-2 pharmaceutical composition.

62. The method of claim 61, wherein said higher total weekly dose of IL-2 is an amount of said multimeric IL-2 pharmaceutical composition equivalent to a total weekly dose of IL-2 of about 1833 μg to about 3300 μg, and wherein said lower total weekly dose of IL-2 is an amount of said multimeric IL-2 pharmaceutical composition equivalent to a total weekly dose of TL-2 of about 1100 μg to about 2567 μg, and wherein said lower total weekly dose of IL-2 is lower than said higher total weekly dose of IL-2.

63. The method of claim 62, wherein said higher total weekly dose of IL-2 is an amount of said multimeric IL-2 pharmaceutical composition equivalent to a total weekly dose of IL-2 of about 1833 μg to about 2567 μg, and wherein said lower total weekly dose of IL-2 is an amount of said multimeric IL-2 pharmaceutical composition equivalent to a total weekly dose of IL-2 of about 1100 μg to about 1833 μg, and wherein said lower total weekly dose of IL-2 is lower than said higher total weekly dose of IL-2.

64. The method of claim 63, wherein said higher total weekly dose of IL-2 is an amount of said multimeric IL-2 pharmaceutical composition equivalent to a total weekly dose of IL-2 of 2567 μg, and wherein said lower total weekly dose of TL-2 is an amount of said multimeric IL-2 pharmaceutical composition equivalent to a total weekly dose of IL-2 of about 1833 μg.

65. The method of claim 62, wherein said therapeutically effective dose of said anti-HER2 antibody is about 4.0 mg/kg on day 1 of said treatment period and about 2.0 mg/kg for each dose thereafter.

66. The method of claim 62, wherein said higher total weekly dose of IL-2 is administered as a single dose or is partitioned into a first series of equivalent doses that are administered according to a two-, three-, four-, five-, six- or seven-times-a-week dosing schedule, and wherein said lower total weekly dose of IL-2 is administered as a single dose or is partitioned into a second series of equivalent doses that are administered according to a two-, three-, four-, five-, six- or seven-times-a-week dosing schedule.

67. The method of claim 27, further comprising an interruption in said two-level dosing regimen of IL-2, said interruption comprising a time period off of IL-2 administration between said first time period and said second time period of said two-level dosing regimen of IL-2.

68. The method of claim 67, wherein said interruption has a duration of about 1 week to about 4 weeks.

69. The method of claim 27, wherein said treatment period comprises one or more subsequent cycles of said two-level IL-2 dosing regimen that is initiated about 1 week to about 4 weeks following completion of a first cycle of said two-level IL-2 dosing regimen or completion of any subsequent cycle of said two-level IL-2 dosing regimen, wherein said anti-HER2 antibody is administered throughout said treatment period.

70. The method of claim 27, wherein said IL-2 is recombinantly produced IL-2 having an amino acid sequence for human IL-2 or a variant thereof having at least 70% sequence identity to the amino acid sequence for human IL-2.

71. The method of claim 70, wherein said variant there of is des-alanyl-1, serine 125 human interleukin-2.

72. The method of claim 27, wherein said anti-HER2 antibody comprises at least one human constant region.

73. The method of claim 27, wherein said anti-HER2 antibody is selected from the group consisting of 4D5, 520C9, an antigen-binding fragment of 4D5, and an antigen-binding fragment of 520C9.

74. The method of claim 73, wherein said anti-HER2 antibody is 4D5 or a humanized, chimeric, or human form thereof.
